(19) United States
(12) Patent Application Publication
(10) Pub. No.: US 2012/0021061 A1
(43) Pub. Date: Jan. 26, 2012

(54) MEDICAL AND NUTRITIONAL FORMULATIONS

(75) Inventors: Ralf-Christian Schlothauer, Tauranga (NZ); Jonathan McDonald Counseil Stephens, Hamilton (NZ)

(73) Assignee: Comvita New Zealand Limited, Paengaroa, Te Puke (NZ)

(21) Appl. No.: 13/141,395

(22) PCT Filed: Dec. 23, 2009
(86) PCT No.: PCT/NZ09/00302

§ 371 Date: Aug. 9, 2011

(30) Foreign Application Priority Data
Dec. 24, 2008 (NZ) NZ581616
Nov. 2, 2009 (NZ) NZ572475

(51) Int. Cl. A61K 35/64; A61P 37/04; A61P 29/00; A61P 31/00

(52) U.S. Cl. 424/537

(57) ABSTRACT

Compositions, methods of production and uses are described to maintain or increase the medical and/or nutritional potency of honey or honey analogues primarily by manipulating or fortifying the phenolic content of the honey or honey analogue in the composition.

### TECHNICAL FIELD

The application relates to medical and nutritional formulations. More specifically, the application relates to medical and nutritional formulations that utilise and control concentration of phenolic compounds to maintain and/or maximise potency.

### BACKGROUND ART

Over the last 40-50 years bacteria have become increasingly resistant to commonly used antibiotics. As a result many infections previously readily cured by antibiotics are now difficult or impossible to treat so (Finch, R. G. (1998) Antibiotic resistance. Journal of Antimicrobial Chemotherapy 42, 125-128). Given this, empirical screening of chemical entities for antimicrobial activity represents an important strategy for the development of novel drugs. Natural products in particular have been a rich source of antimicrobial agents, that in general are associated with low levels of toxicity, and in many cases have a fairly broad spectrum of activity (Silver, L. and Bostian, K. (1990) Screening of natural-products for is antimicrobial agents. European Journal of Clinical Microbiology & Infectious Diseases 9, 455-461). A natural product that has received significant attention due to its anti-bacterial action is honey. Although honey has been used for the treatment of respiratory infections and for the healing of wounds since ancient times (Moellering R C. (1.995). Past present and future antimicrobial agents. American J Medicine, 1995; Supp 6A 11S-18S; Jones H R. Honey and healing through the ages. In Honey and Healing. ed Munn P A and Jones H R. 2001; 1-4. Cardiff, IBRA) it was not until the late 20th century, as a result of the increasing resistance of micro-organisms to antibiotics that research studies began to document the anti-bacterial activity of honey against a number of pathogens (Allen K L. Molan P C. Reid G M. (1991) A survey of the antibacterial activity of some New Zealand honeys. Journal of Pharmacy & Pharmacology. 43(12):817-22; Willix D J. Molan P C. Harfoot C G. (1992) A comparison of the sensitivity of wound-infecting species of bacteria to the antibacterial activity of manuka honey and other honey. Journal of Applied Bacteriology. 73(5):388-94). While the majority of honeys have been shown to have anti-bacterial activity, manuka honey, a honey produced by bees from the flowers of the manuka bush (Leptospermum scoparium) have been shown to possess the highest levels of anti-bacterial activity (Molan P C. The antibacterial activity of honey. 2. (1992). Variation in the potency of the antibacterial activity. Bee World 73: 59-76) and to be active against a range of pathogens including Staphylococcus aureus, coagulase-negative Staphylococci, Enterococci and Pseudomonas aeruginosa (Cooper R A. Molan P C. Harding K G. (1999). Antibacterial activity of honey against strains of Staphylococcus aureus from infected wounds. Journal of the Royal Society of Medicine. 92(6):283-5; Cooper R A, Hales E, Molan P C. (2002). The efficacy of honey in inhibiting strains of Pseudomonas aeruginosa from infected burns. J Burn Care Rehabil 23: 366-70; Cooper R A, Molar P C, Harding K G, (2002). Honey and gram positive cocci of clinical significance in wounds. J Appl Microbiol; 93: 857-63; V. M. French, R. A. Cooper and P. C. Molan. (2005). The antibacterial activity of honey against coagulase-negative staphylococci Journal of Antimicrobial Chemotherapy 56, 228-231). Indeed today manuka honey is a well accepted and established clinical treatment for infection associated with wounds and burns, where it has been shown to have both anti-infective and wound healing properties (Cooper 1999; Molan P C. Potential of honey AM J Clin Dermatol 2001; 2; 13-19; A T Ali, M N Chowdhury, M S al Humayyd. (1991 Inhibitory effect of natural honey on Helicobacter pylori. Trop Gastroenterol).

In addition to its use for the treatment of wounds it has also been shown that manuka honey has antibacterial activity against the gastric pathogen H. pylori, the causative agent of gastritis and the major predisposing factor for peptic ulcer disease, gastric cancer and B-cell MALT lymphoma (N Al Somal K E Coley, P C Molan and B Hancock. (1994). Susceptibility of helicobacter pylori to the antibacterial activity of manuka honey. Journal of the Royal Society of medicine 1994; 87; 9-12; Soto M S. Reddy S G. (1999) Graham D Y. Osmotic effect of honey on growth and viability of Helicobacter pylori. Digestive Diseases & Sciences. 44(3):462-4; Osato M S. Reddy S G. (1999) Graham D Y. Osmotic effect of honey on growth and viability of Helicobacter pylori. Digestive Diseases & Sciences, 44(3):462-4). Indeed a number of in vitro studies have shown that concentrations of manuka honey as low as 5-10% (v/v) can inhibit the growth of H. pylori (Soma) 1994, Osato 1999, Mitchell 1999). This finding is of particular interest given that over recent years resistance to currently available antimicrobial agents against H. pylori has increased dramatically leading to an increasing number of treatment failures (L. Fischbach; E. L. Evans. (2007) Meta-analysis: The Effect of Antibiotic Resistance Status on the Efficacy of Triple and Quadruple First-line Therapies for Helicobacter pylori Aliment Pharmacol Ther.; 26(3)343-357). Indeed, in some populations, the level of resistance to clarithromycin, one of the major antibiotics used in the treatment of H. pylori, has been reported to be as high as 30-40% in some countries and is commonly associated with treatment failure (Josette Raymond, Christophe Burucoa Olivier Pietrini Michel Bergeret Anne Decoster Abdul Wann, Christophe Dupont and Nicolas Kalach (2007) Clarithromycin Resistance in Helicobacter pylori Strains Isolated from French Children: Prevalence of the Different Mutations and Coexistence of Clones Harboring Two Different Mutations in the Same Biopsy helicobacter Volume 12 Issue 2 Page 157-163). Resistance to metronidazole, a second antibiotic commonly used in the treatment of H. pylori infection has also been reported to be high (30%-40% in US and Europe and >80% some countries of the developing world), although in some cases in vitro resistance does not translate into eradication failure (Raymond 2007; Elvira Marvic, Silvia Wittmann, Gerold Barth and Thomas Henlel (2008) Identification and quantification of methylglyoxal as the dominant antibacterial constituent of Manuka (Leptospermum scoparium) honeys from New Zealand Mol. Nutr. Food Res. 2008, 52, 000-000). Given this environment, alternative treatment approaches are of interest.

While the antimicrobial activity of honey has been reported to include osmolarity, acidity, hydrogen peroxide and plant-derived components, more recent studies have shown that osmolarity, acidity and hydrogen peroxide activity cannot account for all of its activity, and that enhanced activity may be due to phytochemicals found in particular honeys, including manuka honey (Molan 1992). For example Cooper et al. (Cooper 1999) in a study of the antibacterial activity of honey against Staphylococcus aureus isolated from infected wounds showed that the antibacterial action of honey in infected wounds does not depend wholly on its high osmolarity, and suggested that the action of manuka honey stemmed partly from a phytochemical component (Cooper 1999).

Until recently the identity of these phytochemicals in manuka honey remained unclear, however in 2008 a study by Marvic et al reported that the pronounced antibacterial activity found in manuka honey directly originated from a chemical compound, methylglyoxal (MGO). In this study six samples of manuka honey were shown to contain over 70 times higher levels of methylglyoxal (up to 700 mg/kg) than that found in regular honeys (up to 10 mg/kg) (White, J. W., Schepartz, A. I. and Subers, M. H. (1963) Identification of Inhibine, Antibacterial Factor In Honey, as Hydrogen Peroxide and Its Origin in a Honey Glucose-Oxidase System. Biochimica Et Biophysica Acta 73, 57-).

Floral Markers

As noted above, phytochemicals are thought to have an important role in relation to activity. Honeys have been known for some time to include a variety of phenolic compounds, flavonoids and abscisic acid. A selection of prior art on this point includes the following documents:

Ferreres et al 1996 (Ferreres et at Natural occurrence of abscisic acid in heather honey and floral nectar. J. Agric. Food Chem. 1996 44, 2053-2056) describes tests done on heather honey to find two non-flavonoid components as the main constituents being two isomers of abscisic acid. The corresponding flower nectar from which the honey is derived were also found to contain both isomers as the main constituents. This document notes that the abscisic acid isomers were not detected in other monofloral honey samples so Ferreres suggests that abscisic acid may be used as a marker for heather honey.

Gheldof et al June 2002 (Nele Gheldof, Xiao-Hong Wang and Nicki J Engeseth (2002) Identification and Quantification of Antioxidant Components of Honeys from Various Floral Sources. J. Agric, Food Chem 2002 50, 5870-5877) describes tests completed on honeys for antioxidant capacity and phenolic content. Antioxidant content was found to be proportional to phenolic content and darker honeys such as buckwheat were found to have high antioxidant capacities. This application suggests that the phenolic content of honey may be used as an indicator of honey origin.

Gheldof et al 2002a (Neie Gheldof and Nicki J Engeseth (2002) Antioxidant Capacity of Honeys from Various Floral Sources Based on the Determination of Oxygen Radical Absorbance Capacity and inhibition of in Vitro Lipoprotein Oxidation in Human Serum Samples J. Agric. Food Chem 2002 50, 3050-3055) describes further experimentation completed from the earlier article. The aim in this article was to characterise the phenolics and other antioxidants in the honeys tested. In this article the authors found that honeys have similar types of antioxidants but different amounts of phenolic compounds. The author concluded that the phenolics were significant to antioxidant capacity but not solely responsible. Examples of antioxidant materials noted included proteins, gluconic acid, ascorbic acid, hydroxymethylfurfuraldehyde and enzymes such as glucose oxidase, catalase and peroxidase. Barberan et al 1993 (Francisco A. Tomas-Barberan, Frederico Ferreres, Cristina Garcia-Viguera, and Francisco Tomas-Lorente (1993) Flavonoids in honey of different geographical origin. Z Lebensm Unters Forsch 196:38-44) describes analysis of flavonoids in honey. The authors of this article found that flavonoids were incorporated into honey from propolis, nectar or pollen and that honeys from the northern hemisphere tended to show higher degrees of propolis based flavonoids while equatorial and Australian based honeys were largely devoid of propolis based flavonoids. South American and New Zealand honeys contained flavonoids associated with propolis.

Yao et al 2003 (Lihu Yao, Nivedita Datta, Francisco A. Tomas-Barberan, Federico Ferreres, Isabel Martos, Riantong Singanusong (2003) Flavonoids, phenolic acids and abscisic acid in Australian and New Zealand Leptospermum honeys. Food Chemistry 81 (2003)159-168) describes the use of measuring flavonoid, phenolic acid and abscisic acid content in Australian and New Zealand honeys as a method of authenticating honey floral origins. The authors found that Australian jelly bush honey included myricetin, luteolin and tricetin as the main flavonoids. Phenolics were found to be primarily gallic and coumaric acids along with abscisic acid. By contrast New Zealand manuka honey contained quercetin, isorhamnetin, chrysin, luteolin and an unknown flavanin. The main phenolic compound was found to be gallic acid. In addition, almost three times the amount of abscisic acid was found in New Zealand manuka honey as Australian jelly bush honey.

Barberan et al 2001 (Francisco A Tomas-Barberan, Isabel Martos, Federico Ferreres, Branka S Radovic and Elke Anklam (2001) HPLC flavonoid profiles as markers for the botanical origin of European unifloral honeys. J Sci Food Agric 81485-496) describes how the phenolic profiles of 52 honeys from Europe were analysed. The different honeys were found to have different markers with different characteristics and UV spectra. Different markers however were found to be present in several honeys rather than being specific to one species. For example, abscisic acid was found in heather honey, rapeseed, lime tree and acacia honeys.

As should be appreciated from the above, a variety of experiments have been undertaken to determine characterising compounds in honeys. Knowledge exists that honey contains antioxidant activity and that this may be attributable to compounds such as flavonoids, phenolic acids and abscisic acid. What should also be apparent from the above is that different studies have found that these compounds are present in a variety of honeys and that the amount present and the types of compound present may be a misleading measure of the honey origin due to their variation and lack of correlation between plant and honey. For example, abscisic acid is found in a variety of different honeys from plant species but the quantities vary substantially even between samples from the same source.

The authors of the above documents do not consider whether honey age has any influence on the composition of the various compounds analysed.

Methoxylation

Most dietary polyphenols have very poor bioavailability due faster metabolic breakdown of hydroxyl groups as opposed to methoxyl groups. Methoxylated phenolics here are highly resistant to human hepatic metabolism (Wen, X., Walle, T. (2006a) Methylation protects dietary flavonoids from rapid hepatic metabolism. Xenobiotica 36: 387-397) and also have much improved intestinal transcellular absorption (Wen, X., Walle, T. (2006b) Methylated flavonoids have greatly improved intestinal absorption and metabolic stability. DrugMetab. Dispos. 34: 1786-1792). The methylated flavones show an approximately 5- to 8-fold higher apparent permeability into cells which makes them much more bio-available. The higher hepatic metabolic stability and intestinal absorption of the methylated polyphenols make them more favourable than the unmethylated polyphenols for use as potential cancer chemo-preventive agents. The determination of metabolic stability of four methylated and their corresponding unmethylated flavones with various chemical structures all of the tested methylated flavones, showed much higher metabolic stability than their corresponding unmethylated analogues.

It should be appreciated from the above that it would be useful to have a means for adjusting the level of medical and/or nutritional potency of honey. Since plants from which honeys are derived contain key compounds with medical and nutritional potency, it should further be appreciated that methods of manipulating plants to enhance key compound levels would be useful. It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the embodiments described herein will become apparent from the ensuing description that is given by way of example only.

### BRIEF DESCRIPTION OF DRAWINGS

Further aspects will become apparent from the following description which is given by way of example only and with reference to the accompanying drawings in which:

FIG. 1 shows a graph illustrating the phenolic profile of monofloral manuka, kanuka, and other honeys harvested in New Zealand and aged naturally for up to ten years;

FIG. 2 shows a graph illustrating the correlation between the sum of the principal phenolic components and methylglyoxal in monofloral manuka honey harvested in New Zealand and naturally aged;

FIG. 3 shows a graph illustrating the presence of selected phenolic compounds in plant nectar for four different plants used in honey production;

FIG. 4 shows a graph illustrating the sum of phenolic markers and MGO in manuka pollen compared to related manuka honey;

FIG. 5 shows two pie charts illustrating the key phenolic compounds in manuka honey and manuka plant nectar;

FIG. 6 shows two pie charts illustrating the key phenolic compounds in kanuka honey and kanuka plant nectar;

FIG. 7 shows a graph illustrating Pseudomonas aeruginosa cultures grown and exposed to a range of manuka, kanuka and clover honeys (10% w/v); control represents no honey treatment, clover honey sugar concentration effect, manuka honeys with a range of MGO concentrations, and kanuka honeys with ineffective concentrations of MGO;

FIG. 8 shows a graph illustrating Staphylococcus aureus cultures grown and exposed to a range of manuka, kanuka and clover honeys (10% w/v), control represents no honey treatment, clover honey sugar concentration effect, manuka honeys with a range of MGO concentrations, and kanuka honeys with ineffective concentrations of methylglyoxal;

FIG. 9 shows a graph illustrating the cytokine IL-1β response in relation to various stimuli where #50336 and #50042 are two manuka honeys and the other stimuli are as labelled;

FIG. 10 shows a graph illustrating the cytokine IL-10 response in relation to various stimuli where #50336 and #50042 are two manuka honeys and the other stimuli are as labelled;

FIG. 11 shows a graph illustrating the cytokine TNFα response in relation to various stimuli where #50336 and #50042 are two manuka honeys and the other stimuli are as labelled;

FIG. 12 shows a graph illustrating microassay results for manuka honey with and without peroxide and/or catalase on growth of E. coli strain 0157:H7. Legend: peroxide (4.0; mM top dose)(x); peroxide (4.0 mM)+catalase, (500 U/ml)(+); honey, (▪), honey+peroxide, (4.0 mM)(▴); honey+catalase, (500 U/mL)(▪); honey+peroxide (4.0 mM)+catalase, (500 U/mL)(▴);

FIG. 13 shows a graph illustrating microassay results for manuka honey with and without peroxide and/or catalase on growth of E. coli strain Nissle. Legend: peroxide (4.0; mM top dose)(x); peroxide (4.0 mM)+catalase, (500 U/ml)(+); honey, (▪); honey+peroxide, (4.0 mM)(▴); honey+catalase, (500 U/mL)(▪); honey+peroxide (4.0 mM)+catalase, (500 U/mL)(▴);

FIG. 14 shows a graph illustrating microassay results for clover honey with and without peroxide and/or catalase on growth of E. coli strain 0157:H7. Legend: peroxide (4.0; mM top dose)(x); peroxide (4.0 mM)+catalase, (500 U/ml)(+); honey, (▪); honey+peroxide, (4.0 mM)(▴); honey+catalase, (500 U/mL)(▪); honey+peroxide (4.0 mM)+catalase, (500 U/mL)(▴);

FIG. 15 shows a graph illustrating microassay results for clover honey with and without peroxide and/or catalase on growth of E. coli strain Nissle. Legend: peroxide (4.0; mM top dose)(x); peroxide (4.0 mM)+catalase, (500 U/ml)(+); honey, (▪); honey+peroxide, (40 mM)(▴) honey+catalase, (500 U/mL)(▪); honey+peroxide (4.0 mM)+catalase, (500 U/mL)(▴);

FIG. 16 shows a graph illustrating the effect of a range of MGO concentrations on the viability of H. pylori SS1. The graph shows the number of CFU of H. pylori recovered on CSA selective plates CSA following incubation of H. pylori SS1 in a control broth containing catalase -⋄-; in MGO -▪-; in control honey C -Δ-; in manuka honey M1 --; and in manuka honey M3 -▴-. The results are the mean of 12 independent measurements. The vertical bars represent standard deviations;

FIG. 17 shows a graph illustrating the effect of pH on the antibacterial activity of a range of concentrations of MGO and manuka honey on H. pylori SS1. The graph shows the number of CFU recovered from selective plates CSA following incubation of H. pylori with MGO-▪-; MGO adjusted to pH 9.0 -□-; manuka honey M1--; and manuka honey M1 adjusted to pH 9.0 The results are the mean of 12 independent measurements. The vertical bars represent standard deviations;

FIG. 18 shows a graph illustrating the percentage of methoxylated phenolic compounds in a set of naturally aged Leptospermum scoparium (manuka) and Kunzea ericoides (kanuka) honeys;

FIG. 19 shows a graph illustrating the incubation of manuka honey with tannase and resulting increase in free phenolic compounds;

FIG. 20 shows a graph illustrating the relative concentration change of MGO in monofloral manuka honey when mixed with other honeys and aged naturally for six months;

FIG. 21 shows a graph illustrating the ratio of observed recovery of MGO and the sum of methoxylated phenolic components in a manuka honey subject to a range of water dilutions compared to the expected recovery;

FIG. 22 shows a graph comparing paired samples illustrating the effect of moderate heating on the concentration of phenolic compounds and MGO in manuka honey, 25% clover honey and 25% rewarewa blends with the same manuka honey. % concentration change represents increase of described component after 50 days treatment relative to initial concentration; and,

FIG. 23 shows a graph comparing paired samples illustrating the effect of acidification and storage at room temperature on the concentration of phenolic compounds and MGO in manuka honey. % concentration change represents increase of described component after 50 and 200 days of storage.

### DETAILED DESCRIPTION

The instant application broadly relates to maintaining and/or maximising the medical and nutritional potency of honey by use of the finding that phenolic compounds in honey are a key driver of honey potency. As should be appreciated, the same findings in relation to honey may be applied to honey analogue compositions as well and hence this specification encompasses both options.

Finding the above synergies was surprising as this goes against recent publications which suggest that methyl glyoxyl (MGO) is the primary compound and those which have found abscisic acid to be a major factor.

A further finding by the inventors was the fact that the free phenolic compounds concentrations change over time in the honey and in response to other factors such as heat and dilution. This change in concentration over time was unexpected and may be a reason why earlier trials looking at phenolic compounds were unsuccessful or gave mixed or inconsistent results.

Also contrary to the art was the inventors finding that in fact phenolic compounds in plant nectar (as opposed to pollen or other measures) was highly correlated to the levels found in honey sourced from the same plants. As noted above, the art does not teach of a correlation between plant nectar phenolic compound levels and that observed in honey and therefore concludes that phenolics are not a useful measure. The art also does not recognise the influence of phenolic compounds on medical potency. In contrast, particularly when age is taken into consideration, phenolic compounds are highly correlated between plant nectar and honey. This finding has meant that it is possible for the inventors to measure a wide range of factors in honey related to honey properties and value well beyond that speculated in the art of only origin.

The exact mechanism behind why the phenolic levels vary in honey overtime is not certain however the inventor understands that these phenolic entities are initially carried by a tannin molecule(s) present in the nectar, and as the honey ages naturally the phenolic molecules are released due to degradation of the tannin body and the matrix associated with a large organic molecule with both hydrophobic and hydrophilic binding sites. The best researched comparison for such aging is the development of flavour and aroma in red wines due to the release of phenolic groups from tannin bodies.

The same result of an increase in MGO concentration over time for honeys that include MGO e.g. manuka honey, was also measured by the inventors although other processing steps could be used to adjust MGO concentration and not adjust phenolic concentration hence a different mechanism of release appears to occur for MGO. Prior art suggests that this may be due to conversion of DHA into MGO as a natural reaction process within the honey and that this reaction is sensitive to age like phenolics as well as other influences e.g. heat and acidification.

The improved healing effects or potency are in part thought to be due to these phenolic compounds working alone or with other properties in the honey to confer multiple stages of healing. The different stages described further below in detail are an antimicrobial phase, an immune stimulation phase and an anti-inflammatory phase. All of these aspects are understood by the inventors to contribute to potency of the honey or honey analogue in medical and nutritional applications.

For the purposes of this specification the term ‘phenolic compounds’ and grammatical variations thereof refers to phenolic acids, phenolic salts, phenolic esters and related polyphenolic compounds.

As will be become apparent from the description below and examples, phenolic compounds of the present invention may be in free form or in a complexed form or a mixture thereof.

The term ‘free’ in the context of phenolics refers to phenolic compounds being in a readily detectable form.

The term ‘complexed’ in the context of phenolics refers to phenolic compounds being carried in a tannin molecule or otherwise not detectable, for example as a result of in vivo phenolic self condensation or precipitation reactions occurring as a result of honey bees dehydrating nectar.

The term ‘honey analogue’ refers to a composition essentially containing only the osmolarity and acidic properties of honey. Typically this is a sugar based solution. The closest equivalent naturally produced honey to a honey analogue is clover honey.

The term ‘manipulated’ refers to adapting or changing a naturally occurring honey or honey analogue to suit a desired end effect.

The term ‘fortified’ refers to adding a compound or compounds to the honey or honey analogue composition of the present invention to suit a desired end effect.

The term ‘artificial’ or grammatical variations thereof refers to altering a honey or honey analogue from a naturally occurring state, in the present invention, typically to achieve a greater level of efficacy.

The term ‘potent’, ‘potency’ and grammatical variations thereof refers to an enhanced medical effect.

According a first embodiment, there is provided a composition including honey or a honey analogue wherein the honey or honey analogue is artificially manipulated and/or fortified to include at least 5 mg/kg of tannin derived phenolic compounds.

Preferably the composition includes at least 5-10,000 mg/kg of tannin derived phenolic compounds.

In the above embodiment, the phenolic compounds may be methoxylated. As noted above, the prior art teaches of useful properties attributable to methoxylated compounds. The inventors have found that honey which includes methoxylated compounds exhibits useful medical and nutritional effects. By way of example, the inventors have analysed the phenolics prominent in manuka (Leptospermum spp.) and kanuka (Kunsea spp.) and a large number of these phenolics are methoxylated at one or more points of their phenol or acid group. Compounds such as gallic or benzoic acid are present mainly in their methoxylated form such as methoxybenzoic acid, methoxygallic acid, methyl syringate, methoxyphenyllactic acid or syringic acid. Methoxylation is therefore a major feature of the phenolics that are prominent in the above species which are acknowledged to have a higher medical and so nutritional activity. The Inventor's findings in combination with the art mean that effects envisaged for medical and nutritional applications include:
Greater bioavailability due to the methoxylated compounds be able to enter the cell faster;
Longer bioavailability due to the methoxylated compounds having a much longer half life within cells to scavenge free radicals;
Phase II enzyme Induction properties.

For honey wound dressing applications, the methoxylated compounds are also likely to have a much longer half life within wound exudate as they are not rapidly degraded.

Methoxylation also results in much longer lived molecules once they are in the cell.

Also unexpectedly, the inventors have found that methoxylated compounds are well tolerated by the human cells (low toxicity) but not by bacterial or fungal cells which is highly advantageous in treating microbial infections.

In a further embodiment, methoxylated phenolics may represent greater than 10% wt total phenolic content in the composition. In one embodiment the content may be greater than 20% wt. In a further embodiment the content may be greater than 30% wt.

In a yet further embodiment, methoxylated phenolics are present at a level greater than 150 mg/kg in the composition.

The phenolic compounds may be selected from the group consisting of: phenyllactic acid, methoxylated phenyllactic acid, methoxylated benzoic acids, syringic acid, methyl syringate, isomeric forms of methyl syringate, and combinations thereof.

The methoxylated derivatives of benzoic acid may be selected from the group consisting of: 2-methoxybenzoic acid, 4-methoxybenzoic acid, trimethoxybenzoic acid and combinations thereof.

In one embodiment the total phenolic content may be measured indirectly by determining the sum of phenyllactic and 2-methoxyphenyllactic acids and derivatives particularly hydroxylated analogues, illustrated below:

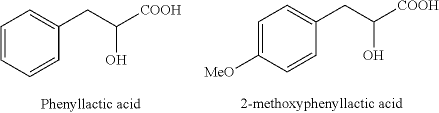

In a young honey these compounds are understood by the inventors to typically account for more than three-quarters of the principal phenolic components. The inventors have found that, with no other influences other than age, honey tend to show an increase in predominance of benzoic acid compounds and their derivatives.

Preferably, the methoxylated derivatives of benzoic acid noted above are: 2-methoxybenzoic acid, 4-methoxybenzoic acid and isomers of trimethoxybenzoic acid as shown below:

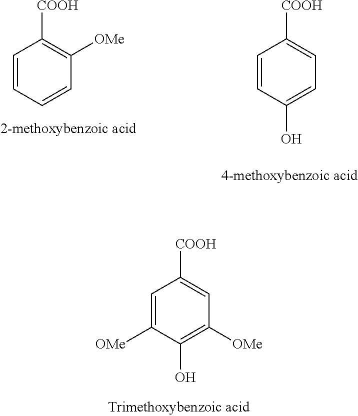

Hydroxylated benzoic acid derivatives (salicylic acid and 4-hydroxybenzoic acid) are also of interest although are present in less significant concentrations.

Preferably, the third group of the principal phenolic components noted above include syringic acid and methyl syringate:

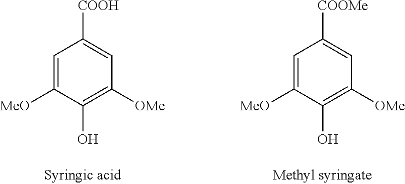

These components are present as two isomers that are diagnostic and differentiate manuka and kanuka honeys.

In a further embodiment, the phenolics may also include a suite of other compounds allied with the tannin matrix in honeys. These range from relatively simple molecules such as gallic acid and methoxylated derivatives, abscisic acid, cinnamic acid, phenylacetic acid and methoxylated and hydroxylated derivatives, and methoxyacetophenone; to complexed polyphenolic molecules such as ellagic acid. A range of these molecules are illustrated below

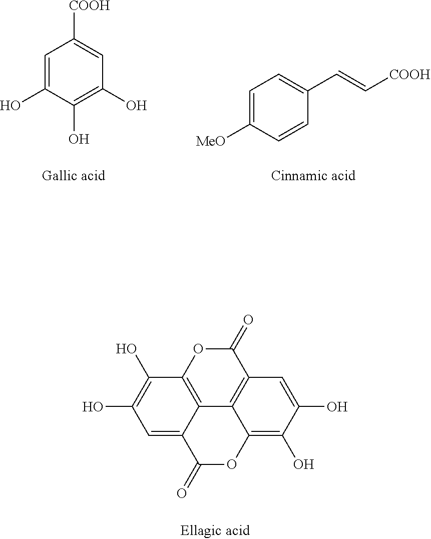

The composition may include a blend of honey or honey analogues. Honey or honey analogue compositions may be selected and/or blended as described above in order to obtain a concentration of phenolic compounds ranging from 5 mg/kg to 10,000 mg/kg or higher depending on the preferred application.

In one embodiment, the honey or honey analogue may be aged for a time period of at least 1 year. Aging may occur for up to 10 years although the most variation in the inventors experience is observed in the first 5 years of aging.

In a further embodiment, the composition may be processed by addition of heat. As should be appreciated, heat can be undesirable due to the production of unwanted hydroxymethylfurfuraldehyde (HMF) compounds hence, use of heat needs to be carefully controlled. In addition, heat appears to increase MGO content if present which exacerbates an antimicrobial phase which may also not be desired. Preferably, the temperature for heating may be less than 50° C. More preferably, the temperature may be less than 40° C.

In a further embodiment, the composition may also be manipulated and/or fortified with further compounds selected from the group consisting of: tannase enzyme, an aqueous dilution agent, commensal bacteria, commensal fungi, flavonoid sources, phenolic compounds from other sources, complexed phenolics, anti-microbial agents, synthetic anti-inflammatory agents, MGO, acidifying agent, and combinations thereof. These compounds may be added to adjust the honey or honey analogue potency and help accentuate one or more phases of healing.

The composition may also be fortified with phenolic compounds. Preferably, the honey or honey analogue may be fortified with phenolic compounds sufficient to result in a concentration of between 5 mg/kg to 10,000 mg/kg or higher of phenolic compounds in the honey or honey analogue. Phenolic compounds may be in free form or in complexed form such as being bound in a tannin complex.

Phenolic compounds added to the honey or honey analogue may be derived from other plant species. For example, the phenolics may be derived from olive leaf extract and in particular the compounds, tyrosol, hydroxytyrosol and oloreupein. Another example may be to use an aloe vera extract and/or a green tea extract.

In one embodiment, the honey or honey analogue may be fortified with methoxylated phenolic compounds.

In one embodiment the aqueous dilution agent may be water. Water is understood to potentially breakdown tannins and release free phenolic compounds present in the honey.

In a further embodiment, the honey or honey analogue may be mixed with fungal material. The inventor's have found that fungal material, for example yeasts, spores, fungal cellular compounds, may have a significant influence on the degree of immune stimulation caused by the honey, particularly when the honey is placed on a wound. Compounds have been identified by the inventor's in high immune stimulation honeys that are commonly associated with fungal cellular material. More specifically, the fungal cellular material may include complex carbohydrate compounds associated with the cell wall of fungal material. Such compounds may be isolated and mixed into the honey or honey analogue composition of the present invention to manipulate the wound healing effects of the composition, particularly for immune stimulation applications. An unexpected result noted by the inventors was that these fungal derived compounds also appear to have a synergistic effect on immune stimulation. As may be appreciated, honey often contains LPS material in the form of cell wall debris, primarily from bacteria in the natural environment. LPS is known to have an immune stimulatory effect that is measurable and reproducible. Experiments undertaken by the inventor's identified that a similar immune stimulatory effect may be observed between LPS and the high fungal material containing honeys, yet the fungal material containing honeys required nearly 200 times less concentration than LPS to acquire the same stimulatory action as LPS. As may be appreciated, honey containing immune stimulation properties may be useful in at least wound dressing applications where the normal innate wound healing process needs to be stimulated in order to treat for example, a chronic wound.

In one embodiment, the fungal material is added (fortified) into the honey. In an alternative embodiment, the honey may be fermented with yeast for a period of time to generate the fungal material. In this embodiment, the fermentation process may be stopped using heat and/or irradiation.

The composition may be formed into a wound dressing by further manufacture into formulations selected from: a cream, an ointment, a gel, a putty, a fibre dressing with the honey impregnated into or around the fibre, a fibre dressing with the honey enclosed within one or more fibre layers, and combinations thereof.

As may further be appreciated, the composition may readily be adjusted to accentuate different phases of healing, for example: to accentuate a first anti-microbial phase or accentuate a third anti-inflammatory phase.

According to a second embodiment, there is provided use of the composition substantially as described above in a wound dressing.

According to a third embodiment, there is provided use of the composition substantially as described above in a nutritional supplement.

According to a fourth embodiment there is provided a method of maintaining or increasing the medical and/or nutritional potency of a honey or honey analogue composition by the steps of:
(a) selecting one or more honeys or honey analogues;
(b) artificially manipulating and/or fortifying the honey or honey analogue to increase the concentration of at least one tannin derived phenolic compound in the honey(s) or honey analogue(s) to a level of 5 mg/kg or higher.

In the above method, step (b) may result in an increase in the amount of phenolic compounds in the honey or honey analogue to a level of between 5-10,000 mg/kg or higher.

Preferably, the phenolic compound concentration may include at least 10% wt methoxylated phenolic compounds. In one embodiment the concentration includes at least 20% wt methoxylated phenolic compounds. In a further embodiment the concentration includes at least 30% wt methoxylated phenolic compounds

Preferably, methoxylated phenolic compounds are present in the honey or honey analogue at a concentration greater than 150 mg/kg.

The phenolic compounds may be selected from the group consisting of: phenyllactic acid, methoxylated phenyllactic acid, methoxylated benzoic acids, syringic acid, methyl syringate, isomeric forms of methyl syringate, and combinations thereof.

The methoxylated derivatives of benzoic acid may be selected from the group consisting of: 2-methoxybenzoic acid, 4-methoxybenzoic acid, trimethoxybenzoic acid and combinations thereof.

Manipulation and/or fortification methods may include blending of different honey types and/or analogues.

Manipulation and/or fortification methods may include aging the honey or honey analogue for a time period of at least 1 year.

Manipulation and/or fortification methods may also include heating the honey or honey analogue. In this embodiment, the temperature for heating may be less than 50° C. Preferably, the temperature is less than 40° C.

Manipulation and/or fortification may include adding tannase enzymes to the honey or honey analogue. In one embodiment, the concentration of methoxylated phenolic compounds is increased by the step of adding tannase enzymes to the honey or honey analogue. It is understood that tannase enzymes may work to breakdown tannin complexes in the honey and release phenolic compounds including methoxylated phenolic compounds in the honey.

Manipulation and/or fortification may include adding an aqueous diluent such as water to the honey or honey analogue. Adding a diluent has been found by the inventor's to alter the tannin phenolic equilibrium and results in the release of additional free phenolic compounds into the composition thereby increasing the concentration of tannin based phenolics in the composition.

Manipulation and/or fortification may include fortifying the dressing with phenolic compounds. In one embodiment, the honey or honey analogue is fortified with phenolic compounds sufficient to result in a concentration of between 5 mg/kg to 10,000 mg/kg or higher of phenolic compounds in the honey or honey analogue.

In one embodiment, the honey or honey analogue may be fortified with methoxylated phenolic compounds.

In a further embodiment the honey or honey analogue may be fortified with methylglyoxal compound.

In a further embodiment the honey or honey analogue may be acidified.

In a further embodiment, the honey or honey analogue composition may be fortified by the inclusion of fungal material. Preferably, the fungal material includes complex carbohydrate compounds associated with the cell wall of fungal cells.

As should be appreciated, combinations of the above processing steps may also be used without departing from the scope of the present invention.

The method above may also involve the step of forming the manipulated and/or fortified composition into a wound dressing by further manufacture into formulations selected from: a cream, an ointment, a gel, a putty, a fibre dressing with the honey impregnated into or around the fibre, a fibre dressing with the honey enclosed within one or more fibre layers, and combinations thereof.

According to a fifth embodiment there is described a method of treatment of a wound by application of a wound dressing containing a honey or a honey analogues wherein the honey or honey analogue has been artificially manipulated and/or fortified to include at least 5 mg/kg of tannin derived phenolic compounds and wherein, on application to a wound, the composition induces three phases of healing including:
(a) an anti-microbial phase;
(b) an immune stimulation phase; and,
(c) an anti-inflammatory phase.

According to a sixth embodiment there is described the use of honey or a honey analogue based composition that has been artificially manipulated and/or fortified to include at least 5 mg/kg of tannin derived phenolic compounds in the manufacture of a wound dressing for the treatment of a topical wound on an animal in need thereof and wherein, on application to a wound, the composition induces three phases of healing including:
(a) an anti-microbial phase;
(b) an immune stimulation phase; and,
(c) an anti-inflammatory phase.

The inventors understand that at least in medical applications, the healing action may be broken into three phases as noted above. These different phases are counterintuitive i.e. immune stimulation/anti-inflammatory conferring opposing effects. The sequence and cascade of these phases appears to be key in the way honey is such an effective agent.

The first phase (anti-microbial) is characterised by the anti-microbial action of the composition. The dressing lowers the pH and elevates osmolarity in the wound area which stresses microbes, particularly bacteria that may be present. Release of hydrogen peroxide as the honey dilutes in the environment also further stresses microbes present. Hydrogen peroxide is produced as water in the environment reacts with glucose to form gluconic acid and hydrogen peroxide catalysed by glucose oxidase. Additional factors that also influence this first anti-microbial stage are the content of methylglyoxal (MGO) in the honey, presence of phenolic compounds in the honey and conversion phenolic compounds from a complexed form into a free form within the honey or honey analogue. The anti-microbial phase is understood to include actions selected from the group consisting of: lowering of the pH, elevation of the osmolarity in the wound area, release of hydrogen peroxide, slowing microbial growth, delaying the onset of microbial growth, stopping microbial growth, killing existing microbes, and combinations thereof.

Whilst the inventor is aware that the medical and nutritional compositions of the invention have anti-microbial effects generally, microbes known to be specifically affected include gram positive bacteria such as Bacillus spp, Staphylococcus spp, Listeria, as well as gram negative species such as Salmonella spp, Pseudomonas spp, E. coli and combinations thereof. In one alternative embodiment, the microbial challenge may be of a fungal origin such as fungi and yeast for example from Candida species.

The immune stimulation phase is understood to include production of pro-inflammatory cytokines selected from the group consisting of: TNFα, IL-1, IL-1β, IL-6, IL-10, 10F-α, and combinations thereof.

The immune stimulation phase is also understood to include debriding action associated by an elevation of MMP protease enzyme activity. The debriding action results in sloughing of dead cellular and foreign matter from the wound. This may be caused in part by the same characteristics as the first anti-microbial phase and may happen after the first phase or happen concurrently with the first phase.

This second phase has particular importance for treatment of recalcitrant or chronic wounds that remain unhealed overtime. This immune system stimulation can ‘kick start’ the hosts immune system and therefore break the chronic healing system dynamics transforming the chronic wound to an acute but progressing wound.

One finding by the inventors is that a relatively young honey with more phenolic compounds bound in complexed form appears to have a greater immune stimulation effect than an aged honey with a greater number of free phenolics. By way of example, the immune stimulation phase may therefore be accentuated by use of a young honeys, fortification with complexed phenolic compounds and/or by minimising heat, age, dilution and acidification of the honey during processing.

The anti-inflammatory phase is understood to include one or more actions selected from the group consisting of: reduction in inflammation, an inhibition of proteolytic tissue degrading enzymes (MMP-proteases), reduction in the levels of free radicals (quenching of peroxide levels), an increase of glutathione levels, induction of phase II enzyme inducer activity, and combinations thereof.

The protease enzyme inhibition noted above includes inhibition of MMP proteins selected from: elastase, gelatinase, keratinase, and combinations thereof.

It should be appreciated that the above three phases of activity are not obvious in view of their obvious clash i.e. immune stimulation and anti-microbial versus anti-inflammatory. However, the different effects and the timing of their importance appears to be critical to the success of the healing process.

In one embodiment, the wound dressing may be manipulated and/or fortified to accentuate the anti-microbial phase. For example, MGO content may be increased by fortification or instead, where MGO is present in the honey naturally, the amount of MGO may be increased by use of heat and/or use of acid.

In a further embodiment, the wound dressing may be manipulated and/or fortified to accentuate the immune stimulation phase. For example, fungal material such as compounds from fungal cell walls may be added.

In an alternative embodiment, the wound dressing may be manipulated and/or fortified to accentuate the anti-inflammatory phase. For example, reduce antimicrobial effects and fortify with phenolics or use aged honey with increased phenolics or add tannase to increase methoxylated phenolic concentration.

In the inventor's experience, the transition between the different phases of healing is defined for the first phase and second phase by the concentration hydrogen peroxide, the concentration of MGO and the pH of the environment at the interface between the medical or nutritional composition and the area being treated.

The transition to the third anti-inflammatory phase of wound healing is understood to be characterised by the concentration of phenolic compounds. In one embodiment, the transition may be characterised by the concentration of methoxylated phenolic compounds. The concentration of phenolic compounds is understood to be proportional to the reduction in inflammation, inhibition of proteolytic tissue degrading enzymes (MMP-proteases), reduction in the levels of free radicals (quenching of peroxide levels), an increase of glutathione levels, induction of phase II enzyme inducer activity, and combinations of these mechanisms. An advantage found by the inventor is that the above combination avoids an excessive inflammatory response from the first and second phases. It is understood that the methoxylated phenolic compounds may play an important part in the various phases of healing and were these compounds not present, the medical and nutritional potency would potentially be lower.

As noted above, the honey or honey analogue compositions produced via the above methods may also be fortified with further agents added to influence the different stages of healing.

In one embodiment, methylglyoxal (MGO) may be added to the honey or honey analogue or instead produced by heating or acidifying a honey that already contains some MGO and thereby liberating more free MGO in the honey. In this embodiment, MGO may be added or increased to a concentration in the composition or dressing of 10-2000 mg/kg. It should be appreciated that the MGO added may be synthetic or naturally derived.

As may be appreciated, an aim of adding MGO may be to enhance the first phase of healing described above. As noted above, studies using synthetic MGO show that these compounds give a strong anti-microbial response and if too much MGO is used, it is toxic or at least may be harmful to the wound healing process as the MGO may overwhelm the normal cell glyoxalase system to detoxify MGO. In the present invention, the presence of phenolic compounds are understood to temper the response and therefore prevent toxic effects for example by their free radical scavenging ability. As a result, MGO may be added with less risk than might otherwise be the case.

Also the inventor's quite unexpectedly found that MGO was negatively correlated to causing inflammation. The inventors conducted an experiment to determine whether increasing MGO concentrations also increase the ability of honey with the MGO to prevent or slow neutrophils from making superoxides (a part of the inflammatory response). Quite unexpectedly, the amount of MGO made no difference at all in the neutrophil process and the greatest effects were found primarily for honeys rich in phenolic compounds. These results further illustrate how MGO may be associated with a first anti-microbial phase of healing but that MGO is at best only weakly associated with a second immune stimulation phase.

In a further embodiment, commensal bacteria or fungi may be added to the honey or honey analogue composition. In one embodiment above the commensal bacteria or fungi may be probiotic bacteria or fungi. It is known in the art that probiotics may assist a host's immune system. Both live probiotics and inactivated probiotics are known to provide an immune stimulation effect.

The art explains that the effect of adding commensal bacteria or fungi is based on cell wall glycoproteins and other cell constituents reacting with immunological receptors of the host. In the case of beneficial probiotics, the response is not signified by exacerbated inflammation as might be expected by a stimuli of LPS but an immune stimulation that creates an improved response to further subsequent challenges to the host e.g. by pathogens.

Probiotic bacteria or fungi may also be useful in breaking down the tannin complex and thereby increasing the number of free phenolic compounds in the honey. By way of example, Lactobacillus plantarum, a beneficial micro-organism that inhabits the human gut has been shown to degrade tannin complexes by catalysing the hydrolysis of ester and depside linkages in hydrolysable tannins into individual phenolic units thus freeing the biologically active units for cell absorption.

In a further embodiment, commensal bacteria or fungi may be live or attenuated. Preferably, the live or attenuated bacteria or fungi noted above stimulate the immune system. In this case, use of live or attenuated bacteria or fungi also is to avoid an exacerbated inflammation response and instead prompt an immune stimulation that creates an improved response to further subsequent challenges to the host e.g. by pathogens.

In a further embodiment, the compositions described above may be fortified with various flavonoid sources or extracts of flavonoid sources including: berries, green tea, cruciferous species extracts including cabbage and broccoli, olives, olive leaf, bark, propolis, pollen, and combinations thereof.

Optionally, other antimicrobial agents may be added to the composition such as silver particles, iodine and antibiotics, particularly for wound dressing applications.

Optionally, other down regulating agents may be added to the composition such as ibuprofen or diclofenac HCl.

As should be appreciated, combinations of these additional agents may be used without departing form the scope of the embodiments described herein.

Optionally, non-phenolic containing honeys may also be used e.g. clover honey. This may be done to accentuate the anti-microbial phase. This may also be completed to shift the proposed chemical equilibrium towards faster tannin breakdown and therefore release free phenolics into the composition.

The honey or honey analogue may also be characterised by enhanced osmotic pressure and an acidic pH.

The osmotic activity may be equivalent to the composition having a sugar content greater than 30% M. Lower levels may also be used to stress selected microbes or may only be required owing to synergies with other components in the composition (for example 10% wt). Osmolarity is considered important as this dehydrates microbes present in wounds which slows growth, stops growth or even kills microbes altogether depending on the degree of osmolarity.

The pH level may be between approximately 3.5 and 5. Preferably, the pH is approximately 3.8 and 4.6.

It is envisaged that wound dressing compositions would be used on topical wounds such as cuts, grazes, burns, open wounds, exudating wounds, stitched wounds (e.g. after surgery) and the like. Examples are provided by way of illustration only and should not be seen as limiting.

In preferred wound dressing embodiments, the dressing may be applied and later re-applied as needed during the wound healing process. The dressing may be removed and a fresh dressing re-applied without harm to the wound. As noted above regarding the three phases, the tempering effect of the third phase allows the wound dressing to be able to be re-applied without harm. A further advantage of phenolic compounds being present is that they prevent accumulation of MGO compound in the patient serum.

In one embodiment, the animal that is treated may be a human. In alternative embodiments, the animal may be a non-human.

Preferably, the amount of phenolics included in the compositions of the invention may be varied depending on an individual's body weight and individual metabolism. The dose may also vary dependent on the species of animal treated—for example, a wound dressing may equally be used on humans as on horses, cattle, sheep, dogs and cats. For example, racing horses with wounds may be treated by application of a wound dressing according to embodiments described herein.

As noted above, the compositions and methods described have potential wound healing advantages in part at least due to the multiple phases of healing induced.

A further advantage of the embodiments described herein is that use of phenolics in honey may reduce the stinging sensation or pain on application reported particularly when elevated levels of MGO are naturally present or added to the honey used.

It should be appreciated from the above description that there are provided compositions, uses and methods to maintain and/or maximise the medical and nutritional potency from honey or honey analogue compositions.

Embodiments are now described with reference to various examples illustrating the medical and nutritional properties of the embodiments.

### Example 1

In this example, honey harvested from the indigenous New Zealand shrubs Leptospermum scoparium (manuka) and Kunzea ericoides (kanuka) are used to demonstrate the presence of free phenolic compounds and the way the concentration of these compounds change over time. Manuka and kanuka honeys were chosen to illustrate this effect as they contain relatively high levels of free phenolics and derivative compounds compared to other honey types.

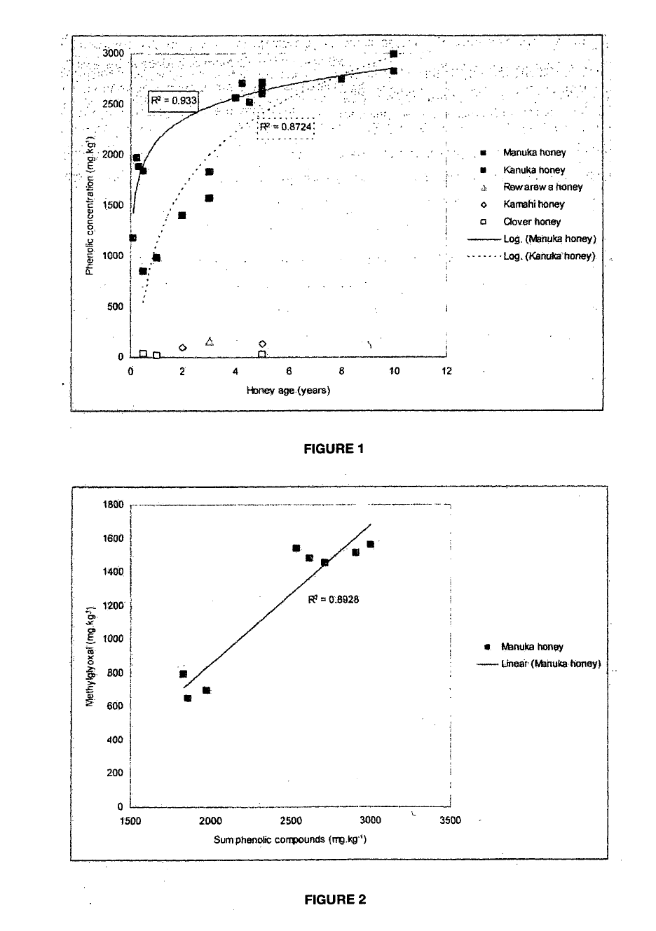

FIG. 1 illustrates the concentration of the free phenolics present in five honey types of different ages. Relatively fresh (<3 months) manuka and kanuka honeys contain approximately 1000 mg·kg⁻¹ of these compounds, whereas in comparison the other honey types of the same age contain considerably less than 100 mg·kg⁻¹. Furthermore as the manuka and kanuka honeys are aged naturally, that is stored at room temperature following extraction from the honey comb, the concentration of the phenolic components increases approximately three-fold over ten years to in the region of 3000 mg·kg⁻¹. However, the increase in free phenolic components' concentration illustrates a logarithmic curve; consequently much of the development of the phenolic profile occurs in the first five years of the honeys storage and aging.

Table 1 below describes the concentrations of these components during the aging process. Whilst these compounds are common to manuka and kanuka honeys, the concentration of some components differ significantly in these honeys.

TABLE 1
The phenolic profile and concentration of principal components mg/kg in monofloral
manuka and kanuka honeys harvested in New Zealand and aged naturally for ten years.

| | | | | | | | | Sum principal | |
|---|---|---|---|---|---|---|---|---|---|
| | | | Phenyllactic | 4-methoxy- | Methoxylated | Methyl | Syringic | phenolic | |
| Honey Type | Age (yr) | n | Acid | phenyllactic acid | benzoic acids | syringate | acid | components | Methylglyoxal |
| Manuka | 0.5 | 3 | 1743 ± 77.5 | 4.8 ± 0.3 | 31.3 ± 4.1 | 19.3 ± 2.4 | 94.2 ± 8.0 | 1893 ± 78 | 714 ± 72.3 |
| | 5 | 3 | 1880 ± 40.0 | 4.9 ± 2.5 | 31.7 ± 3.4 | 310.7 ± 58.5 | 394.8 ± 32.4 | 2622 ± 91 | 1492 ± 45.0 |
| | 10 | 2 | 2001 ± 58.0 | 15.0 ± 4.2 | 33.5 ± 6.4 | 383.5 ± 40.3 | 520 ± 82.0 | 2953 ± 62 | 1538 ± 31.8 |
| Kanuka | 0.5 | 2 | 700.7 ± 26.1 | 93.3 ± 15.5 | 2.3 ± 0.8 | 63.3 ± 8.5 | 103.7 ± 11.9 | 963 ± 20 | 42.4 ± 23.4 |
| | 5 | 2 | 1549 ± 83.4 | 307.0 ± 21.2 | 3.4 ± 1.1 | 336.0 ± 12.7 | 592.5 ± 14.8 | 2788 ± 10.6 | 35.5 ± 26.2 |
| | 10 | 1 | 1680 | 512 | 7.2 | 338 | 554 | 3091 | 17.0 |

Values shown, mean ± standard deviation

The concentration of methylglyoxal in the manuka and kanuka honeys is also listed in Table 1. Manuka honey, derived from Leptospermum scoparium, contains methylglyoxal. As a manuka honey is aged, the concentration of free methylglyoxal also increases in the honey. This increase is understood to be due to a different mechanism to the increase in phenolics owing at least to the way the compounds develop when heated. It is understood by the inventors that the MGO increase may be due to conversion of DHA to MGO.

FIG. 2 illustrates the correlation between the concentration of methylglyoxal and the principal phenolic compounds in a naturally aged manuka honey. Methylglyoxal and total phenolic compounds do no correlate in kanuka honey because the methylglyoxal component is derived from Leptospermum scoparium, and the small amounts of methylglyoxal in the kanuka honeys represent insignificant manuka honey contamination.

### Example 2

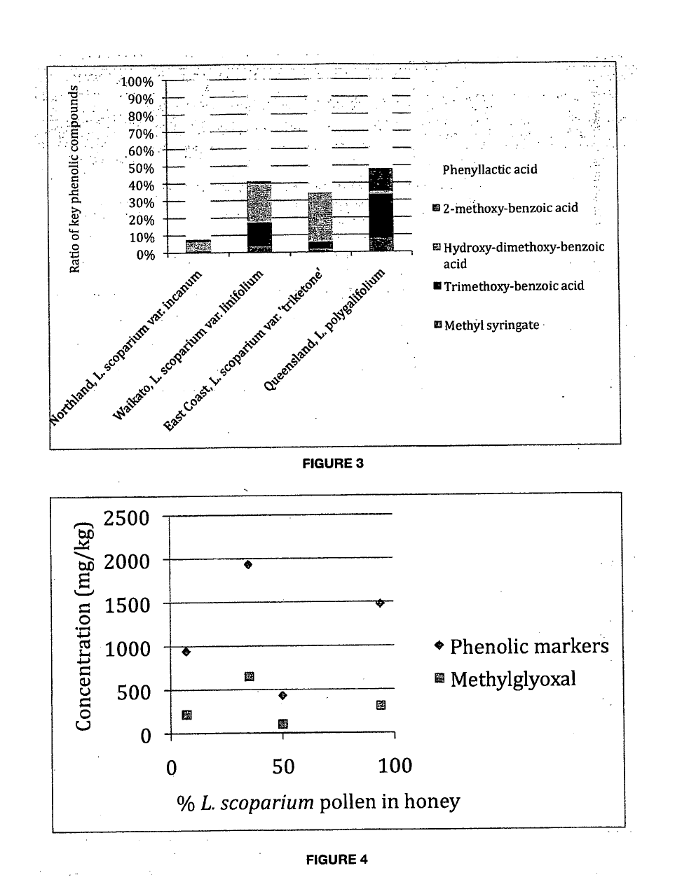

A further illustration of the presence of unique phenolic compounds in plant nectar used for honey manufacture is illustrated in FIG. 3 which shows a comparison between manuka honey produced from Northland, Waikato and East Coast in New Zealand and a sample from Queensland, Australia.

As can be seen in FIG. 3, the ratio of phenolic compounds allows separation by region, and botanic source. The concentration of 2-methoxy-benzoic and tri-methoxy-benzoic acids is significantly elevated in honey derived from Leptospermum polygalifolium in Queensland, Australia. Phenyllactic acid is elevated in honey from Northland, New Zealand where variety is Leptospermum scoparium var. incanum. Elevated tri-methoxy-benzoic acid separates honey sourced from the Waikato wetlands and the East Coast of the North Island, New Zealand.

### Example 3

In this example, tests were completed to confirm the presence of phenolic compounds in plant nectar from which honey is derived.

The phenolic components can be isolated from the nectar of plant varieties and species. Table 3 below illustrates some of the components isolated mg/kg from two distinct cultivars of Leptospermum scoparium, and Kunzea ericoides. All of the phenolic compounds that are present in the honeys are derived from these species and are present in the species' nectar.

TABLE 2
Phenolic components measured in cultivars of Leptospermum scoparium and Kunzea encoides (mg/kg)

| | Phenyllactic | Sum of Methyl | Sum of Syringic | 2-Methoxy- | 4-Methoxyphenyl- | |
|---|---|---|---|---|---|---|
| Plant Variety/Species | Acid | Syringate Isomers | Acid Isomers | benzoic Acid | lactic Acid | Methylglyoxal |
| L. scoparium | 90 | 530 | 8.9 | 9.3 | — | 14 |
| cultivar 1 | | | | | | |
| L. scoparium | 450 | 330 | 8.6 | 11.8 | — | 32 |
| cultivar 2 | | | | | | |
| Kunzea ericoides | 380 | 850 | 14.3 | Trace | 72 | Nil |
| | | | | | | Detected |

Given that the honey bees perform about a ten-fold concentration of the nectar during the conversion into honey it is apparent three of these principal components are relatively more concentrated in the nectar than in the honey. This is evidence of in vivo phenolic self-condensation reactions occurring as the honey bees perform nectar dehydration. Such in vivo self-condensation reactions have been well described in the study of aging in wine (Monagas, M.; Gomez-Cordoves C.; Bartolome, B. 2004. Evolution of the phenolic content of red wines from Vitis vinifera L. during ageing in bottle. Food Chem. 95(3) 405-412). In contrast syringic acid concentration is similar in nectar and fresh honey, indicating this molecule is mostly present as hydrolysable tannin in the nectar and the increased concentration in aged honey may be due to tannin body degradation.

The analysis of nectar components in various glasshouse conditions provides measurement of the plants production of the different components, and secondly production efficiency in different environments. This allows breeding selection to be tailored to fit the intended locations for plantation establishment.

### Example 4

As noted above, plant nectar showed an unexpected and strong correlation to honey in phenolic marker compounds. This finding is contrary to the art which leads away from this correlation. To exemplify why this finding is unexpected, a trial was completed whereby a variety of bee pollen samples were collected and analysed to assess the concentration of a selection of key phenolic markers.

The key phenolic markers were phenyllactic acid, methoxyphenyllactic acid, 2-methoxybenzoic acid, 4-methoxybenzoic acid, syringic acid, methylsyringate, hydroxydimethoxybenzoic acid and trimethoxybenzoic acid.

The resulting concentrations were compared against that measured in honey of the same source to observe whether a correlation exists between the pollen and honey. MGO results were also taken as a further comparative measure.

As shown in FIG. 4, whilst the key phenolic markers were detected in both the pollen and honey, there is no correlation between the two components with a wide scatter of results. In addition, MGO results also showed no correlation between the pollen and honey levels.

This example illustrates that pollen is not a reliable indicator of phenolic levels in honey unlike plant nectar demonstrated in earlier examples.

### Example 5

This example demonstrates further the correlation between key phenolic markers in nectar and honey.

As noted above, pollen phenolic concentration does not correlate well with honey phenolic concentration. In contrast, and as demonstrated above as well, a correlation is observed between plant nectar phenolic concentration and honey phenolic concentration. This example further illustrates this correlation by comparing samples of manuka honey and nectar as well as samples of kanuka honey and nectar.

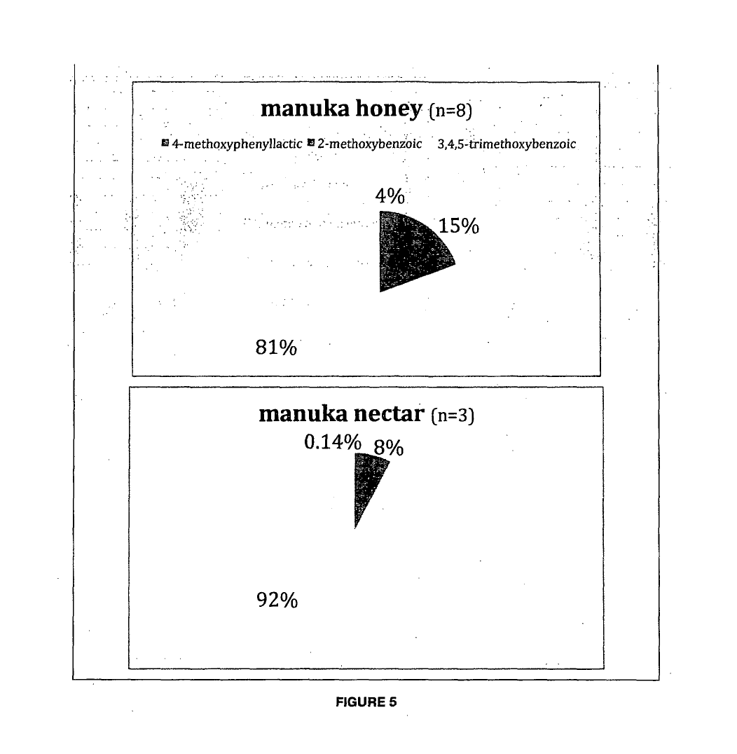

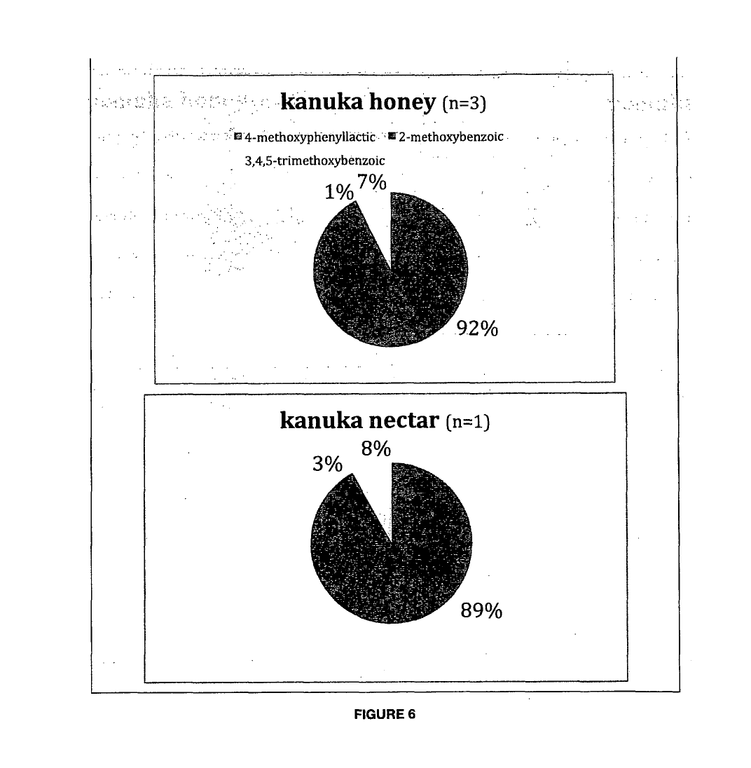

As shown in FIG. 5 and FIG. 6, the comparative concentrations of the three phenolic compounds were highly correlated in the honey and nectar in both manuka and kanuka thereby further illustrating the correlation between these two components.

### Example 6

As noted in the above description, the inventors have established that the wound healing effects of honey have three phases of action. The first phase of action has been found to be an antimicrobial effect. The antimicrobial effect is attributable to a number of known factors such as the honey pH, peroxide activity and high osmolarity of the honey. The inventors have also found that this antimicrobial effect may also be due to the amount of MGO present in the honey (if any) and presence of phenolic compounds.

Experiments are now provided showing the anti-microbial effect that different honeys have and the influence of MGO and/or phenolic compounds.

A number of experiments were performed to determine whether concentration of honey, type of honey or a dilution series was required to demonstrate inhibition or give end point values for inhibition. The experiment was completed over a 16 to 24 hour time period and samples taken and tested for microbial growth as measured by optical density every two hours.

Pure clover samples were used. Those skilled in the art will appreciate that clover honey does not include non-peroxide activity and therefore provides a measure of antimicrobial factors such as pH, peroxide activity and osmolarity influence absent of other effects such as from MGO and phenolics. Samples were also included using various manuka honey samples with activities ranging from a UMF factor of less than 5, approximately 14 and approximately 30 UMF i.e. varying levels of MGO and phenolics. As maybe appreciated from the art, the UMF activity may be attributable to MGO. A control with no honey was also used and several kanuka samples to show any phenolic specific effects i.e. ineffective levels of MGO are present in kanuka honey.

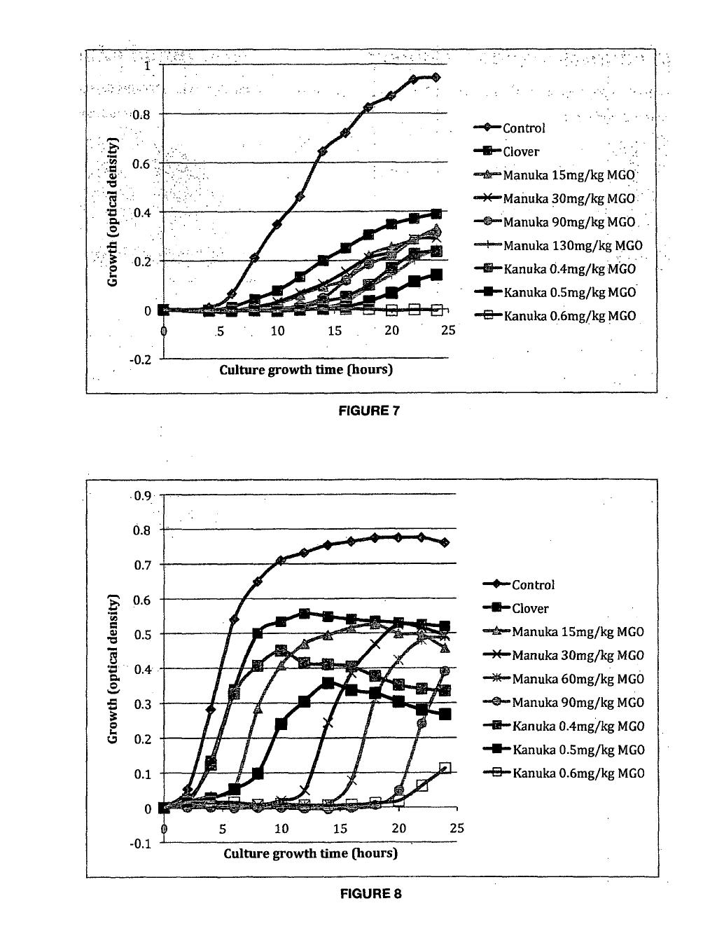

The results found for Pseudomonas aeruginosa (gram negative bacteria) is shown in FIG. 7.

The gram negative bacterial species Pseudomonas aeruginosa is an opportunistic human pathogen that displays multi-drug antibiotic resistance. The growth of this species is inhibited in a linear manner by manuka honeys containing a range of methylglyoxal concentrations; the principal effects are an extension of the lag phase as a dose response with a depression of maximum growth. In contrast kanuka honeys, that contain insignificant concentrations of methylglyoxal that are inadequate to affect growth, inhibit Pseudomonas aeruginosa more effectively by extending the lag phase, reducing maximum growth, and exhibiting the ability to completely inhibit growth throughout the assay period. Therefore the phenolic components contained by kanuka honeys are more effective inhibiting Pseudomonas aeruginosa than the methylglyoxal component of manuka honey.

The results observed above for Pseudomonas auriginosa were also observed by the inventors for other gram negative bacteria including E. coli and Salmonella spp.

The results found for Staphylococcus aureus (gram positive bacteria) are shown in FIG. 8.

The gram positive bacterial species Staphylococcus aureus is also an opportunistic human pathogen that displays antibiotic resistance. The growth of this species is inhibited by manuka honeys and this lag phase inhibition correlates linearly with the methylglyoxal concentration, however there does not appear to be a significant decrease of maximum growth compared to the clover (sugar) control.

The kanuka honeys containing insignificant concentrations of methylglyoxal but significant phenolic compound concentration inhibited Staphylococcus aureus more effectively by extending the lag phase, and reducing maximum growth. The use of artificial honeys or honey analogues seeded with phenolic acid components has shown this effect to be relative to the concentration of phenolic acids found in this honey type.

Similar results have also been found by the inventors for other gram positive bacteria including Listeria and Bacillus spp.

Further experiments have been completed by the inventors using Candida albicans yeast which also showed a similar effect to that noted above, namely that phenolics have an important part to play in anti-microbial activity.

The above results show that different honeys have an anti-microbial effect. The key difference is the rate and degree of inhibition that occurs. For honeys that do not have non-peroxide activity, the delay in growth is still observed due to factors such as pH, osmolarity and peroxide content. The anti-microbial effect can however be significantly enhanced by use of a honey that includes phenolic compounds. MGO contributes to the anti-microbial effect but is not the driving factor.

It should be noted that the inhibition of contaminating strains is important in terms of reducing the total bio-burden below the critical colonisation count for wounds. The wound is bombarded with the release of bacterial antigens and toxins that prevent healing while infected. The immune system is constantly kept in a state of inflammation because of the high bacterial stimulus. The rate of wound breakdown and rebuilding of wound tissue is an unfavourable balance keeping the wound chronic and recalcitrant. Only when this cycle is broken healing can progress. The inhibition of infecting bacteria is the important first step.

In addition, the degree of inhibition may be significant in allowing time for second and third phases of healing to occur. By way of example, honey applied to a wound prevents microbial growth allowing time for immune stimulation and later anti-inflammatory effects to take place. It is the inventors understanding that if microbial growth were not inhibited in the way seen in the above graphs for manuka honey, second and third phases may not occur, or may occur at a slower rate than would be desired for medical or nutritional applications.

### Example 7

In this example, the way honey elicits a second phase of immune stimulation reaction is described.

An experiment was completed using three honey samples, the first being a high UMF manuka honey containing 1002 mg of MGO/kg, a moderate UMF manuka honey containing 649 mg of MGO/kg, a clover honey containing minimal MGO/kg and pure MGO alone.

Results were compared against a negative control with cells alone as a positive control and PHA as the inflammatory agent.

Human blood was collected from six donors and peripheral blood mononuclear cells were separated by Ficoll gradients. Donor bloods were processed in pairs. The experiments tested the effect of the honey on the blood cells and the levels of cytokines produced.

Supernatents from cells incubated with varying honey and MGO concentrations were collected and assayed for a number of pro and anti-inflammatory cytokines. Cytokines are molecules secreted by cells that are involved in communication between cells. Cytokines bind to specific receptors on cell surfaces and the signals then created can alter cellular functions. The cytokines tested in this study were IL-1β, IL-10 and 10F-α.

Cytokines were measured using a biorad, bioplex suspension array system.

Clover honey was used as a control as it is known to contain minimal non-peroxide activity and no MGO.

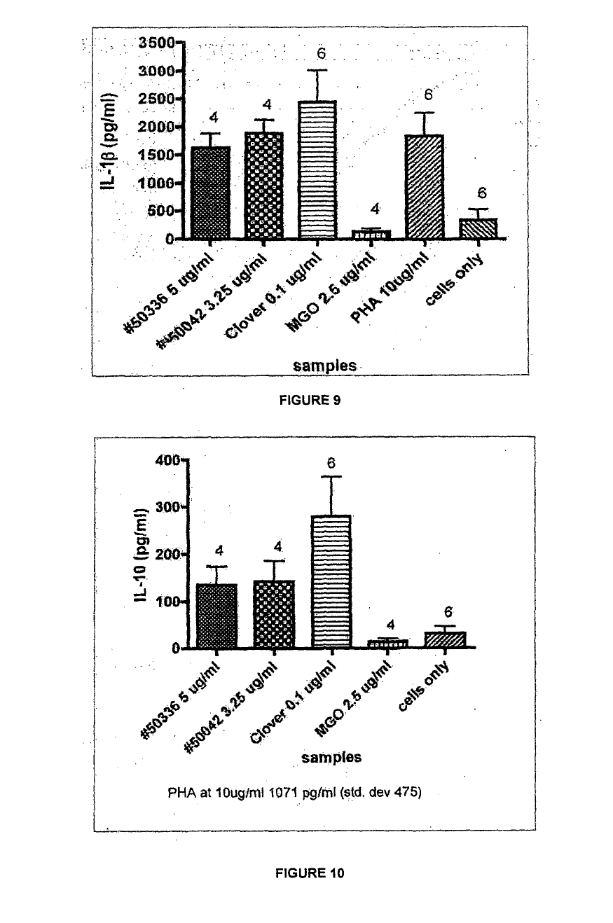

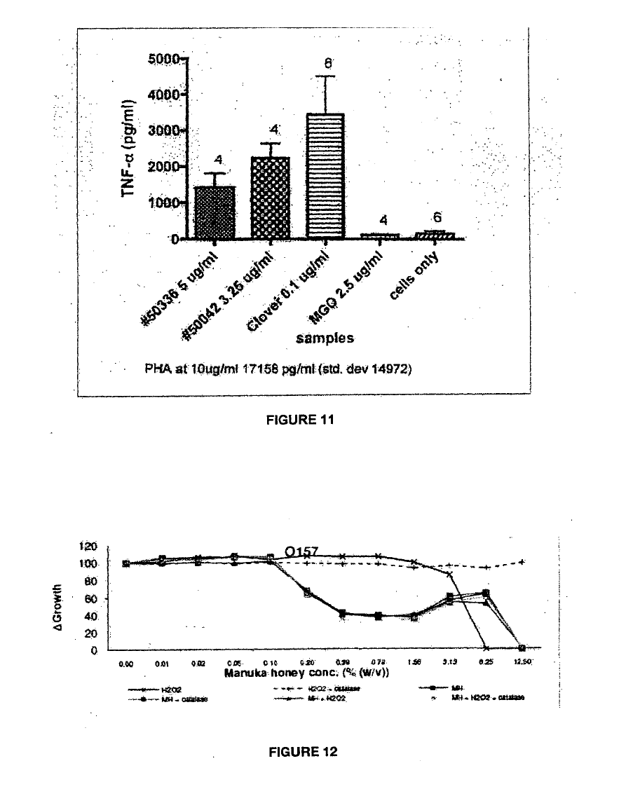

The results found are shown in FIGS. 9 to 11.

As noted from the above graphs, the effects of the three honeys tested on the product of pro and anti-inflammatory cytokines was unrelated to the concentration of methylglyoxal in the products as evidenced by the results obtained with pure MGO compared to those in the presence of honey.

The above results also show that 10F-α production from manuka and clover honey was similar. 10F-α is a pro inflammatory cytokine and is involved in up regulation of IL-1β production. Both of these cytokines have a role to play in the pathogenesis of inflammatory disease. IL-1β levels from cells stimulated with either of the honey types were similar.

Methylglyoxal has no effect on the product of either of these cytokines.

IL-10 values were not affected by MGO and the values from clover samples were slightly higher than those from manuka honey samples. IL-10 is a cytokine that dampens the inflammatory response in vivo.

Also unexpectedly, manuka honey being rich in phenolic compounds produced a pronounced effect at even very low concentrations. This differs to the art e.g. Tonks et. al. which suggests little differentiation in effect between honeys. Since manuka is rich in phenolic compounds and in particular, methoxylated phenolic compounds, this unexpected effect is likely to be attributable to the phenolic compounds since MGO was shown to have no effect on cytokines.

The results obtained in this study prove that honey and in particular, phenolic compounds in honey are responsible for an immune stimulation effect on blood and blood cells. The effect appears to be associated with several cytokines including IL-1β, IL-10 and TNF-α. This effect is independent of the MGO content of the honey.

### Example 8

A further example is provided illustrating the phase II inducer activity of honey rich in hydrolysable tannins. As noted above, phase II induction is in the inventors experience understood to be part of the anti-inflammatory third phase of healing.

Buckwheat and manuka honeys were tested as these are known to be rich in antioxidant compounds.

The method of testing enzyme induction was taken from the art (Fahey et al ‘The Prochaska Microtiter Plate Bioassay for Inducers of NQO1’ Methods in Enzymology, Vol. 382 p 243-258, 2004).

The tests were completed in 2000, 2007 and in 2008. Results found are shown in Table 3 below.

TABLE 3
Multi-Year Honey QRIP Values

| | QRIP (2000) | QRIP (2007) | QRIP (2008) |
|---|---|---|---|
| Honey | (Units/gram) | (Units/gram) | (Units/gram) |
| Dutch Gold; NAO-045 | 2172 | 4762 | 4348 |
| SunBerg | | | |
| Buckwheat (JHU 32) | | | |
| Dutch Gold; NA9-238 BMaid | 1282 | 3333 | 2857 |
| Buckwheat (JHU 3) | | | |
| Manuka | 714 | — | 1695 |
| (Waitemata Honey Co.) | | | |
| (JHU30) | | | |

The above results showed that the phase II induction effect is present as expected in these honeys with high levels of antioxidant compounds. In addition, the results also illustrate the age effect whereby over time compounds responsible for the phase II induction effect become more active.

### Example 9

A further example is now provided illustrating the first phase and third phase of healing action observed by the inventors.

Antimicrobial assays were completed using 96-well microplate analyses, the first column of a 96-well plate loaded with bacterial growth medium supplemented with 25% (w/v) honey and/or 8.0 mM peroxide. Multiple plates were used, each containing different ingredients (honey/peroxide). Ingredient (honey/peroxide) concentrations were halved at each dilution from the maximum concentration for each ingredient, forming a two-fold dilution series, resulting in 11 dilutions each containing eight replicate wells (50.0 pL). Eight replicate control wells containing medium without ingredient were included in the last column of the 96-well plate.

Plates were inoculated with an equal volume (50.0 pL) of bacteria at a density of 103 cells/mL (+/−1000 U/mL catalase), thus diluting the concentration of assay ingredients by half, and the optical density (OD) of the plate was immediately measured at a wavelength of 620 nm using a Thermo Multiscan EX 96 well plate reader to determine the blank (zero growth) value. Plates were incubated at 37° C. for 16 h, and then the OD was determined to measure the growth of the cultures.

The effects of the extracts on the growth of the bacteria were compared by converting the OD of the supplemented culture to a percentage of the control, unsupplemented culture, representing increased or decreased growth, respectively, where the magnitude of deviation from the control (100%) was a measure of relative efficacy.

To estimate antimicrobial activity using well diffusion, agar petri dishes were inoculated by spread-plating bacterial suspensions of sufficient density to form a confluent lawn upon overnight incubation. After inoculation the plates were allowed to dry for 1 h, and then holes were bored in the agar using a sterile implement. Honey and/or peroxide were pipetted into the hole to the level of the agar, and the plate was incubated face upwards. Exclusion zones were photographed, and the radius measured at two points for calculation of area of inhibition.

The honeys used in the trial were monofloral manuka honey UMF 20+ and a dark multifloral honey with minimal manuka honey labelled ‘clover honey’ in the tables below. The honey labelled clover did in fact contain a large amount of kanuka honey, hence contained a large number of phenolic compounds but not MGO.

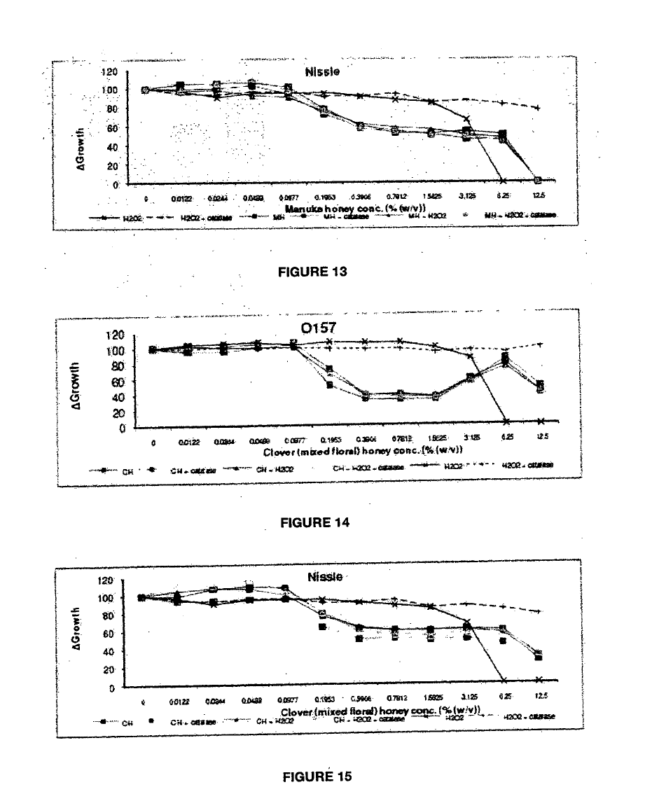

The results found as shown in FIGS. 12 to 15 and in Table 4 below.

TABLE 4
Well diffusion assay comparing manuka honey and clover honey with
addition of peroxide and/or catalase to examine possible contribution of
peroxide to antimicrobial activity, as defined by the size of the inhibition
zone in a lawn of E. coli O157/Nissle.

| | | | Mean |
|---|---|---|---|
| Honey Sample (30% | | | inhibition zone |
| (w/v)) | Peroxide (mM) | Catalase (U/mL) | (n = 3) (mm) |
| Manuka | — | — | 1.1 |
| Manuka | 36.7 | — | 0.6 |
| Manuka | — | 1000 | 1.0 |
| Manuka | 36.7 | 1000 | 1.2 |
| Clover | — | — | 0.0 |
| Clover | 36.7 | — | 1.0 |
| Clover | — | 1000 | 0.1 |
| Clover | 36.7 | 1000 | 0.1 |
| — | 36.7 | — | 3.3 |
| — | — | 1000 | 0.0 |
| — | 36.7 | 1000 | 0.0 |

The above results confirm that honeys have anti-microbial effects. The results also confirm that much lower concentrations of manuka honey are required to achieve anti-microbial effects again showing the influence of phenolic compounds.

The above results were also used to complete a phenolic assay and antioxidant assay. Manuka UMF™ 20+ and control (“clover” (mixed floral)) honey samples were measure their total phenolic content using the Folin assay (Folin, O. and Ciocalteu, V. (1927). J. Biol. Chem. 73, 627-650) modified by (Djeridane, A., Yousfl, M., Nadjemi, B., Boutassouna, D., Stocker, P. and Vidal, N. (2006) Food Chemistry 97, 654-660) as an indication that the manuka honey possessed potential antioxidant compounds.

The manuka UMF™ 20+ and control honeys were subjected to a Ferric Reducing Ability [of Plasma], or Ferric Reducing/Antioxidant Power (FRAP) assay (Benzie, I. F. F. and Strain, J. J. (1996). Analytical Biochemistry 239, 70-76; Bertoncefj, J., Dobersek, U., Jamnik, M. and Golob, T. (2007), Food Chemistry 105, 822-828) to measure “antioxidant potential” to determine whether the honeys should be capable of destroying peroxide and thereby providing an anti-inflammatory effect. This assay compares the ability of the sample to reduce ferric-tripyridyltriazine (Fe TPTZ) complex to the intensely blue ferrous form at low pH with the reducing ability of the powerful antioxidant 6-hydroxy-2,5,6,8-tetramethylchroman-2-carboxylic acid (trolox), a water soluble Vitamin E analogue.

The results found are shown below in Tables 5 and 6.

TABLE 5
Estimation of phenolic content of manuka and clover control honey in
Gallic Acid Equivalents. Data mean of two determinations, each
conducted in triplicate.

| Sample | GAE (mg/kg) |
|---|---|
| Manuka honey UMF ™ 20+ | 842.4 ± 0.3 |
| Clover (control) honey | 656.2 ± 17.9 |

TABLE 6
Estimation of FRAP of 25% (w/v) manuka and clover control honey as
millimolar trolox equivalents. Data mean of two replicates.

| Sample | FRAP (Trolox mM Eq.) |
|---|---|
| Manuka honey | 0.804 |
| Clover (control) honey | 0.701 |

### Example 10

As noted in the above description, the invention has uses for food or nutritional treatments. In this example internal treatment is described. Manuka honey in particular has published antibacterial activity associated with treatment of wounds, but also activity against the gastric pathogen H. pylori. H. pylori is the causative agent of gastritis and the major predisposing factor for peptic ulcer disease, gastric cancer and B-cell Malt lymphoma.

Recent publications in 2007 and 2008 suggest that MGO is the primary antimicrobial agent in manuka honey.

In this example, this assumption of MGO being responsible for antibacterial activity is tested with respect to H. pylori.

Materials and Methods

Bacterial Strains and Growth Conditions

The H. pylori SS1 strain was obtained and initially cultured on Campylobacter Selective Agar (CSA) plates and incubated at 37° C. under microaerobic conditions (10% Co₂) for 48 h. Following culture, the isolates were checked for purity (microscopy, catalase and oxidase), after which SS1 was cultured in Brucella broth supplemented with 5% Fetal Bovine Serum and 1% Vitox under microaerobic conditions with shaking for 24 h.

MGO and Honey Preparations

The effect of 1) methylglyoxal (MGO), density 1.17 g/ml, 2) Honey M1: Manuka honey O50336-10 (MGO content=0.593 mg/g), 3) Honey M2=Honey M1 adjusted to a pH of 9.0, 4) Honey M3=Comvita Wound Care Honey 18+ (MGO content=0.6 mg/g) and 5) Honey C: Control honey (100% Australian Honey, Leabrook Farms, Australia) on H. pylori was determined.

Preparation of MGO and Honey Concentrations

MGO at concentrations of 1, 5, 10, 20, 50 and 100 mg/L were prepared by dissolving an appropriate amount of MGO in Brucella broth. To ensure an equivalent concentration of MGO in the Manuka honeys, the appropriate weight of honey (Honey M1 and Honey M2=6.75 g), (Honey M3=6.67 g) was measured out, after which an equal volume of Brucella broth at 37° C. was added to the honey and this was vortexed to aid mixing. Following vortexing, an equal volume of catalase solution (2 mg/ml) prepared from bovine liver was added to negate the effect of hydrogen peroxide, and this was again vortexed to aid mixing. The total volume was then adjusted to 40 ml using Brucella broth. This resulted in a honey concentration of 16.7% that contained a concentration of MGO equal to 100 mg/L. The control Honey C was prepared in a similar fashion starting with an initial weight=6.71 g). To avoid contamination with lactic acid bacteria that are commonly present in honey (e.g. Leuconostoc citreum and Lactococcus garvieae), preparations of honey M1 and Honey C were filter sterilized through 0.22 pm pore size membrane filters prior to use in the in vitro assay. The Comvita wound care 18+ honey was not filter sterilised as this product undergoes sterilisation on manufacture.

Adjustment of the pH of MGO and Manuka Honey M1

To determine the effect of pH on the activity of MGO and Manuka honey against H. pylori, the pH of MGO and Honey M1 (initially treated with catalase solution) was adjusted to pH 9.0 using 5N sodium hydroxide (NaOH), after which they were filter sterilized.

Assay to Determine the Effect of MGO and Honey on H. pylori

H. pylori were grown in broth culture overnight as described above. Following incubation, cells were washed once and resuspended in Brucella broth to an Optical Density (OD) 600 1.5. Ten pl of the microbial suspension was then inoculated into wells of a 96-well microtiter plate containing 200 pl of broth, supplemented with a range of concentrations of MGO (0, 1, 5, 10, 20, 50 and 100 mg/L), or an equivalent concentration of MGO contained in the Manuka honeys, or the same percentage of control honey. Each of the concentrations of MGO, MGO in the Manuka honeys and the control honey were tested in triplicate. Following addition of H. pylori to the microtiter plates, these were incubated under microaerobic conditions (10% CO₂) overnight. Following incubation the number of viable colonies in the wells was assessed using the drop plate method. Following incubation the number of visible colonies on CSA agar plates were counted and the colony forming units (CFU)/ml calculated. The results from two independent experiments represented as the mean±1 standard deviation of the logarithmic values of CFU/ml for 12 counts (three identical wells, each well counted in duplicate/2 independent experiments).

Results

Effect of Honey and MGO on H. pylori

The results of different concentrations of MGO alone and equivalent concentrations of MGO in three honeys [Manuka honey (M1), Honey M3-Comvita Wound Care 18+ and control honey (C)] on the viability of H. pylori SS1 are shown in Table 6. At concentrations of MGO≧20 mg/L, and control honey concentrations of greater than or equal to 4.2%, the CFU of H. pylori SS1 were reduced, with a concentration of MGO of 100 mg/mL of MGO and control honey at 16.7% showing a fall in CFU units from 1.7×10⁷ to 1.1×10⁴ and non detectable levels (<10³). In contrast Manuka honeys with MGO concentrations greater than or equal to 5 mg/L (honey concentration: 1%) led to a decrease in CFU of H. pylori, with at higher concentrations (50 mg/L and 100 mg/L)<10³ H. pylori being observed (Table 7). Filtration of honey M1 to remove any contaminating bacteria did not appear to affect the activity of the honey against H. pylori SS1 (Table 7).

TABLE 7
Effect of MGO and honey on Helicobacter pylori, viable count (CFU/ml)

| | | | | | | Manuka | Manuka |
|---|---|---|---|---|---|---|---|
| MGO | Honey | | | Control | Manuka | honey M1 | honey |
| Conc* | Conc* | MGO | MGO (pH 9) | honey | honey M1 | (pH 9) | wound care |
| 0 | 0 | 1.7 × 10\hat{ }7 | 1.7 × 10\hat{ }7 | 1.7 × 10\hat{ }7 | 1.7 × 10\hat{ }7 | 1.7 × 10\hat{ }7 | 1.7 × 10\hat{ }7 |
| 1 mg/L | 0.5% | 1.7 × 10\hat{ }7 | 1.6 × 10\hat{ }7 | 1.7 × 10\hat{ }7 | 1.1 × 10\hat{ }7 | 1.4 × 10\hat{ }7 | 1.2 × 10\hat{ }7 |
| 5 mg/L | 1.0% | 1.4 × 10\hat{ }7 | 1.4 × 10\hat{ }7 | 1.5 × 10\hat{ }7 | 7.5 × 10\hat{ }6 | 1.1 × 10\hat{ }7 | 8.1 × 10\hat{ }6 |
| 10 mg/L | 2.1% | 1.2 × 10\hat{ }7 | 1.4 × 10\hat{ }7 | 1.5 × 10\hat{ }7 | 3.8 × 10\hat{ }6 | 7.1 × 10\hat{ }6 | 3.5 × 10\hat{ }6 |
| 20 mg/L | 4.2% | 7.1 × 10\hat{ }6 | 1.2 × 10\hat{ }7 | 1.2 × 10\hat{ }7 | 3.6 × 10\hat{ }6 | 1.5 × 10\hat{ }6 | 2.4 × 10\hat{ }5 |
| 50 mg/L | 8.4% | 7.0 × 10\hat{ }5 | 6.9 × 10\hat{ }6 | 3.5 × 10\hat{ }6 | ND | 1.3 × 10\hat{ }4 | ND |
| 100 mg/L | 16.7% | 1.1 × 10\hat{ }4 | 1.7 × 10\hat{ }6 | ND | ND | ND | ND |

*Conc = Concentration

Effect of pH on the Activity of Honey and MGO Against H. pylori

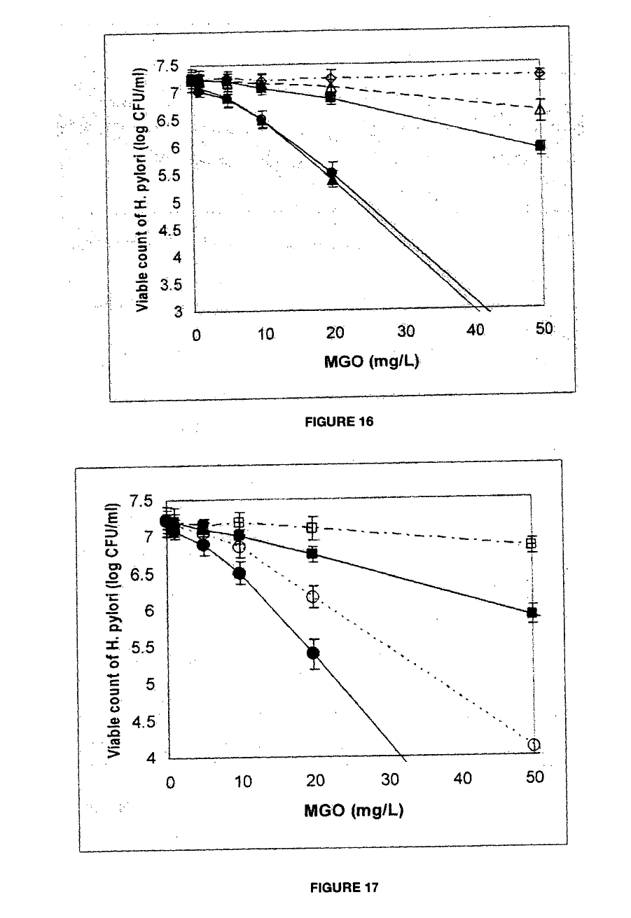

Investigation of the effect of pH on the activity of MGO and manuka honey M1 indicated that the anti-H. pylori activity of MGO and manuka honey M1 was reduced at an alkaline pH as compared with MGO and manuka honey M1 where the pH had not been adjusted (FIG. 16). For example the number of CFU of H. pylori SS1 following exposure to a concentration of 50 mg/L MGO at pH 9 was 6.9×10⁶ as compared with 7.0×10⁵ following exposure to the untreated MGO. The greatest impact however was found at a concentration of 100 mg/L where the effect of MGO at pH 9 on H. pylori viability was significantly reduced (1.7×10⁶) as compared with that in untreated MGO (1.1×10⁴). Investigation of the effect of adjusting the pH of the M1 honey to pH 9 showed that at an ‘equivalent MGO’ concentration of 50 mg/L, the CFU of H. pylori following incubation with M1 at a pH of 9 was 1.3×10⁴ as compared with <10³ in the untreated M1.

The above experiment was designed to also explore the mechanisms of antimicrobial activity of manuka honey against H. pylori. Previous studies identified the major antibacterial substance in honey to be hydrogen peroxide and also demonstrated that it is produced by the enzyme glucose oxidase. Therefore, prior to antibacterial assays, the honey samples were initially treated with catalase, an inhibitor of glucose oxidase, which would eliminate the antibacterial effect from hydrogen peroxide.

The results of the current study confirmed that manuka honey had increased antibacterial activity against H. pylori as compared with other honeys. Interestingly as the concentration of the honey decreased, the osmotic effect of the honey also decreased. For example at a honey concentration of 4.2% (equal to 20 mg/L of MGO) the effect of the control honey became negligible, however the manuka honey maintained a high anti-H. pylori activity with a reduction of CFU of H. pylori to 1% of the original number observed (FIG. 16). These findings are consistent with those in the art. In contrast however, the current study identified that, at lower concentrations, <10% and <5%, the manuka honey had a greater inhibitory effect on H. pylori than US honeys (100% and 60% versus 78% and 33% respectively).

This finding of the inhibitory effect occurring at lower concentrations was irrespective of the presence or absence of catalase and further reinforces that an oxidant effect is not responsible for the killing.

As shown in FIG. 16 the inhibition of H. pylori by MGO alone occurred in a dose-dependent manner with at a concentration of 20 mg/L of MGO (4.2% of manuka honey) the CFU's of H. pylori being 7.1×10⁶ for MGO alone and 3.6×10³ and 2.4×10³ for manuka honeys 1 and 3 respectively. This finding would indicate that other anti-bacterial factors are likely to be present in the manuka honey.

Investigation of the effect of pH on the anti-H. pylori activity of MGO and manuka honey showed that the anti-H. pylori activity of MGO and the manuka honey were both decreased by the more alkaline environment, however the activity of the honey was less affected than that of the MGO alone. This finding suggests that the activity of MGO contained in manuka honey is reduced under high pH while a residual component of the manuka honey maintained good anti-H. pylori activity at a raised pH.

In conclusion, the present study has shown that although H. pylori counts can be reduced to some degree by MGO and control honey, a significantly greater reduction was observed using the manuka honeys as compared with control honey. The demonstrated antibacterial activity appeared to involve a combination of osmolarity, MGO, and more importantly—a phenolic component. These results also illustrate that honey, particularly with phenolic compounds may be used to treat H. pylori.

### Example 11

The above example illustrates use of the compositions of the present invention in terms of H. pylori. In this example, in vivo data is provided further illustrating H. pylori activity.

An experiment was completed feeding manuka honey with a UMF 22 to Helicobacter pylori infected mice at a rate of 2.8 g/kg body weight honey.

The trial model is summarised in Table 8 below.

TABLE 8
H. pylori in vivo trial results

| | Infected | | |
|---|---|---|---|
| Groups | with H. pylori | Treatment post-infection | Number of mice |
| Group 1 | − | Baseline group | 12 |
| Group 2 | + | Control water | 12 |
| Group 3 | + | Sugar solution | 12 |
| Group 4 | + | MGO in sugar solution | 12 |
| Group 5 | + | Manuka honey | 12 |

The results found in FIG. 17 showed that MGO does not reduce bacteria numbers alone but instead whole honey is needed (MGO+phenolics). Manuka honey alone is a treatment that stands out. This ao result is in keeping with the in vitro data collated (see above).

### Example 12

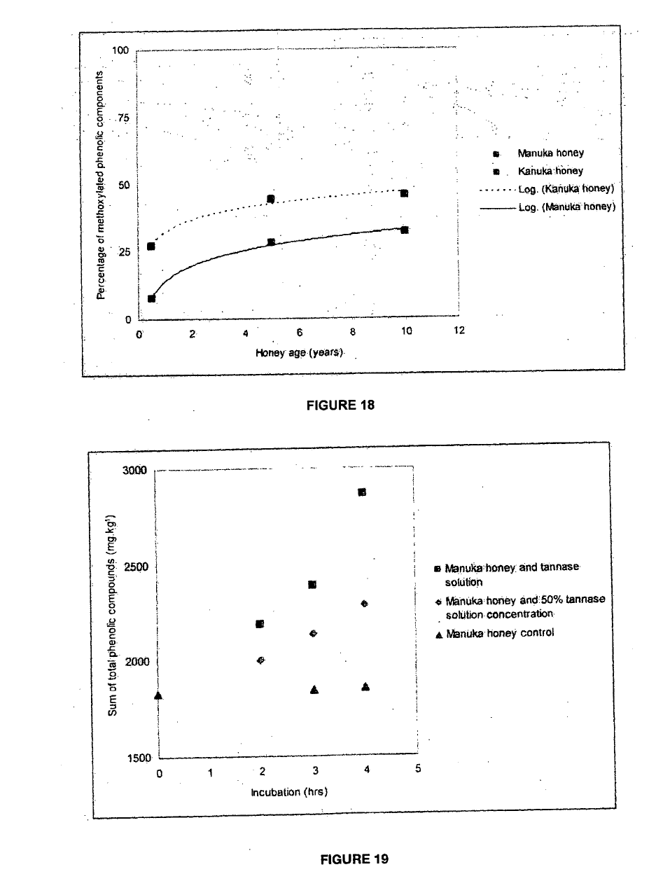

As noted above, methoxylated phenolic compounds are of interest. In this example methoxylated compound profiles are illustrated. The development of the methoxylated phenolic compound profile is similar for naturally aged manuka and kanuka honeys (FIG. 18). In relatively fresh manuka and kanuka honeys the methoxylated components account for approximately 10% and 30% wt of the total free phenolic compounds respectively. That proportion rises to 30% and 45% wt in manuka and kanuka honey respectively in five-year old naturally aged honeys.

Accordingly as the total free phenolic compound concentration increases in these honeys the proportion of methoxylated phenolic compounds also increases. This means the concentration of methoxylated components in five-year old naturally aged honeys is greater than 1000 mg/kg wt⁻¹.

### Example 13

In this example a range of honey samples were analysed to determine the antioxidant levels in the honeys compared to control standards.

Antioxidant activity was determined by the ABTS assay using a spectrophotometric method for antioxidant activity using the ABTS radical assay (expressed as Trolox Equivalent Antioxidant Capacity) based on the method of Miller & Rice-Evans (Miller, N. J.; Rice-Evans, C. A. 1997: Factors influencing the antioxidant activity determined by the ABTS+radical cation assay. Free Radical Research 26(3): 195-199).

All samples were diluted with warm water as required to bring into the appropriate range for the assay.

The antioxidant activities of the various samples are given in Table 9.

TABLE 9
Antioxidant Levels for Honey Samples Tested

| | Antioxidant | |
|---|---|---|
| | activity by | |
| | ABTS assay | |
| | (pmole | |
| | TEAC/100 g) | |
| | | Standard |
| Sample Description | Average | deviation |
| Far North, North Island NZ Honey (Fresh) | 131.4 | 3.7 |
| Far North, North Island NZ Honey (Aged) | 256.2 | 4.0 |
| Bush blend honey from Hokianga, NZ (fresh) | 176.4 | 9.2 |
| Bush blend honey from Hokianga, NZ (aged) | 189.9 | 0.8 |
| Waikato, NZ wetlands Honey (Fresh) | 143.8 | 1.5 |
| Waikato, NZ wetlands Honey (Aged) | 237.7 | 7.5 |
| East Coast, North Island NZ Honey (Fresh) | 153.9 | 4.3 |
| East Coast, North Island NZ Honey (Aged) | 243.7 | 3.4 |
| Kanuka Honey (Fresh) | 178.3 | 1.4 |
| Kanuka Honey (1 Year Old) | 148.5 | 7.4 |
| Kanuka Honey (2 Years Old) | 193.3 | 8.3 |
| Kanuka Honey (3 Years Old) | 239.6 | 11.4 |
| Heated Manuka Honey | 305.1 | 2.5 |
| Clover Honey | 49.7 | 3.2 |
| Rewarewa Honey | 215.9 | −3.4 |
| Standard - 2-methoxybenzoic, 80 mg/kg | 51.8 | 1.3 |
| Standard - phenyllactic acid, 210 mg/kg | 54.6 | 1.3 |
| Standard - methyl syringate, 290 mg/kg | 85.1 | 2.7 |
| Standard - Gallic acid, 700 mg/kg | 1695.4 | 58.3 |
| Standard - Syringic acid, 760 mg/kg | 499.6 | 25.3 |

As can be seen in Table 9, the antioxidant levels increase in honey with age supporting earlier Examples. This effect occurs irrespective of region from which the honey has been collected.

Also noted was that honeys known to have medical activity e.g. manuka honey, had moderate TEAC levels. Conversely, honeys known to have little medical activity e.g. rewarewa honey had higher TEAC counts. This variation in medical activity is understood by the inventors to be attributable to the phenolic levels (total TEAC count), but also the amount of methoxylated phenolic compounds. Manuka honey has been found by the inventors to have a high number of methoxylated phenolic compounds e.g. methoxybenzoic acid and methyl syringate. In contrast, honeys such as rewarewa have been found to contain fewer methoxylated phenolic compounds and more non-methoxylated phenolics such as gallic acid. As noted in the above description, methoxylated compounds appear to have a greater degree of potency.

### Example 14

As noted above, methoxylated phenolic compounds appear to have a greater presence in honeys (and hence nectars from honeys) that are associated with greater medical activity e.g. manuka honey.

A further example is provided below demonstrating the quantity of methoxylated phenolic compounds in a variety of honeys and their comparative levels to further exemplify the presence of these methoxylated compounds in more ‘active’ honeys as opposed to less ‘active’ honeys.

In this example a wide range of honeys were tested using the same criteria to measure the presence and concentration of 2-methoxybenzoic acid as a representative methoxylated phenolic acid. The results found are shown below in Table 10.

TABLE 10
Honey and Methoxylated Phenolic Compound Concentrations

| | | Sample | | 2-Methoxy- |
|---|---|---|---|---|
| | | age | Geographic | Benzoic Acid |
| Honey | Principal floral origin (and possible floral contaminates) | (year) | origin | [mg/kg] |
| Manuka^{a} | L. scoparium var. incanum (Trifolium spp.) | 0.1 | Northland | 32.7 |
| Manuka^{a} | L. scoparium var. incanum (Trifolium spp.) | 0.5 | Northland | 28.9 |
| Manuka^{a} | L. scoparium var. incanum (Trifolium spp.) | 0.9 | Northland | 29.0 |
| Manuka^{b} | L. scoparium var. incanum (hive site not assessed) | 2.5 | Northland | 52.1 |
| Manuka^{b} | L. scoparium var. incanum (hive site not assessed) | 3.5 | Northland | 50.7 |
| Manuka^{b} | L. scoparium var. incanum (hive site not assessed) | 4.75 | Northland | 22.2 |
| Manuka^{b} | L. scoparium var. incanum (hive site not assessed) | 5 | Northland | 14.8 |
| Manuka^{b} | L. scoparium var. incanum (hive site not assessed) | 5 | Northland | 36.3 |
| Manuka^{a} | L. scoparium var. incanum (Trifolium spp., Knightia excelsa) | 0.4 | Northland | 5.7 |
| Manuka^{a} | L. scoparium var. incanum (Trifolium spp. K. excelsa, Kunzea | 0.75 | Northland | 4.3 |
| Manuka^{a} | L. scoparium var. linifolium (Trifolium spp., Weinmannia | 0.25 | Waikato | 22.2 |
| Manuka^{a} | L. scoparium var. linifolium (Trifolium spp., Weinmannia | 0.5 | Waikato | 23.3 |
| Manuka^{b} | L. scoparium var. linifolium (hive site not assessed) | 4 | Waikato | 4.5 |
| Manuka^{a} | L. scoparium var. myrtifolium (Trifolium spp., Knightia excelsa) | 0.5 | Whanganui | 1.2 |
| Manuka^{a} | L. scoparium var. triketone^{d} (Trifolium spp.) | 0.1 | East Coast | 5.9 |
| Manuka^{a} | L. scoparium var. triketone^{d} (Trifolium spp.) | 0.3 | East Coast | 6.4 |
| Manuka^{a} | L. scoparium var. triketone^{d} (Trifolium spp.) | 0.5 | East Coast | 6.4 |
| Manuka^{b} | L. scoparium var. triketone^{d} (hive site not assessed) | 5.5 | East Coast | 1.4 |
| Manuka^{b} | L. scoparium (variety unknown, hive site not assessed) | 1.5 | Unknown | 9.9 |
| Kanuka^{a} | Kunzea ericoides (Trifolium spp.) | 0.1 | Northland | Trace |
| Kanuka^{b} | Kunzea ericoides (hive site not assessed) | 1.5 | Northland | 0.7 |
| Kanuka^{b} | Kunzea ericoides (hive site not assessed) | 2.5 | Waikato | 0.3 |
| Kanuka^{b} | Kunzea ericoides (hive site not assessed) | 3.5 | East Coast | 1.1 |
| Clover^{c} | Trifolium spp. (hive site not assessed) | 1 | South Island | Trace |
| Rewarewa^{b} | Knightia excelsa (hive site not assessed) | 5 | Bay of Plenty | 0.4 |
| Nectar^{e} | L. scoparium var. incanum cultivar, 4 samples | — | Bay of Plenty | 17.7 (8.6) |
| Nectar^{e} | Leptospermum Nichollsil derived cultivar, 2 samples | — | Bay of Plenty | 7.6 (2.1) |
| Nectar^{ef} | Kunzea ericoides, 1 sample | — | Bay of Plenty | 0.5 |

^{a}Samples collected from hive sites;
^{b}Aged samples from drums supplied by apiarists and purchased as designated type;
^{c}Commercially labelled product;
^{d}Unclassified L. scoparium variety that carries an enhanced triketone essential oil profile;
^{e}Nectar samples collected from flowering specimen;
^{f}Qualitative measurement.

As shown in Table 10, the concentration of 2-methoxybenzoic acid is higher in manuka origin honeys than either kanuka, clover or rewarewa derived honeys suggesting methoxylated phenolic compounds may be important to medical efficacy.

### Example 15

As should be apparent from the above examples and description, that phenolic compounds (and to a lesser extent MGO) are of key interest. As illustrated above, the concentration of free phenolic compounds in honey increases over time. It should therefore be appreciated that, to increase the medical and/or nutritional potency of honey or honey analogues, the honey or analogue may be aged.

Artificial development of the phenolic compound profile may also be completed by addition of tannase enzyme as demonstrated below.

Tannin acyl hydrolase EC3.1.1.20 (tannase) activity has been found by the inventors to artificially increase the free phenolic compound content. Addition of the tannase enzyme is understood to degrade the hydrolysable tannin matrix in honey.

An experiment was completed by initially purifying extracellular tannase from a Basidiomycota culture grown on a tannin enriched substrate. The resulting buffered enzyme solution was added to manuka honey and incubated at 40° C. for four hours.

The addition of tannase resulted in a rapid development of the free phenolic compounds in the manuka honey during the 4-hour incubation.

Treatment with two dilutions of tannase solution revealed that the increase in free phenolic compounds in the honey was dependent on the concentration of enzyme as the substrate. The inventors also noted that there was no significant tannase loss of activity in this period (FIG. 19). It is understood by the inventors that the tannase enzyme degrades the ester linkages and glycosidic linkages in the hydrolysable tannin matrix therefore releasing free phenolic compounds in the honey.

### Example 16

Blending of different honeys together and/or addition of water to honey has also been found by the inventors to result in an increase in free phenolic acid concentration.

An experiment was completed blending Leptospermum scoparium (manuka) or Kunzea ericoides (kanuka) honeys with other honey types or dilution of the honey with water to illustrate the effect.

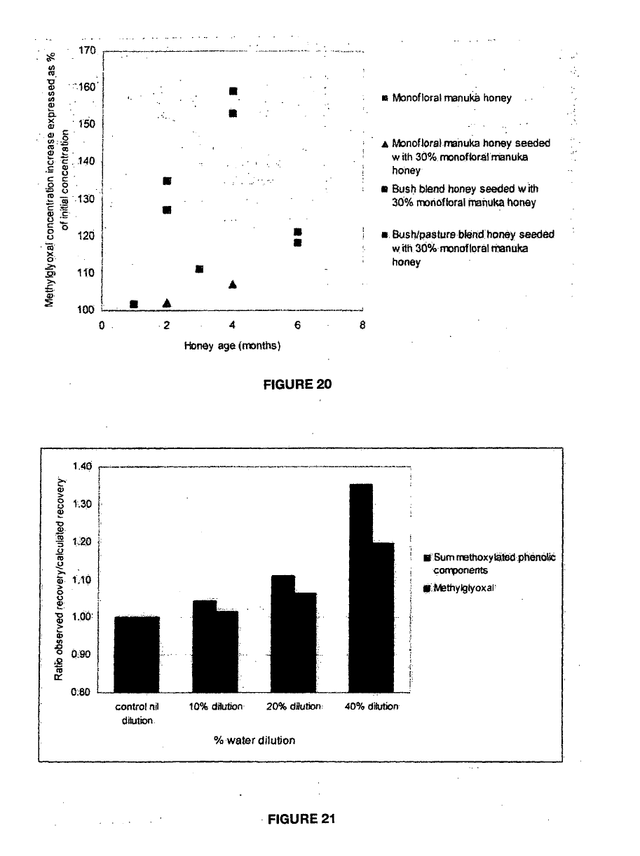

FIG. 20 illustrates the relative change in the concentration of methylglyoxal in monofloral manuka honey that has been aged for six months, the effect of blending two monofloral manuka honeys, compared to the effect of blending manuka honey with bush and pasture honeys.

The results found further illustrate the chemical binding equilibrium understood to exist. When the two monofloral manuka honeys are blended there is little change in the rate of tannin matrix degradation, yet when the concentration is shifted in favour of the complexed compounds by blending pasture or bush honeys with monofloral manuka honey the equilibrium in the honey readjusts and there is a rapid release of methylglyoxal into the honey solution.

More specifically, the concentration of free phenolic compounds in bush/manuka blend honey illustrated in FIG. 20 increased by 63% during six months of natural aging, similar to the increase of methylglyoxal, yet the monofloral manuka honey total free phenolic compound concentration only increased by 24% In the same period. Blending appears to shiftthe equilibrium in favour of greater free phenolic development.

As an additional example the data from a 24-hour aging experiment is presented in FIG. 21. A monofloral manuka honey was diluted with water at a range of percentages, and the recovery of methylglyoxal and methoxylated phenolic components was compared to the expected recovery given the amount of dilution. Diluting resulted in a release of methoxylated phenolic compounds and MGO although to different extents.

Consequently manipulating the dilution of Leptospermum scoparium and Kunzea ericoides honeys by blending with other honey types or diluting with water brings about a more rapid release of the phenolic compounds and methylglyoxal.

### Example 17

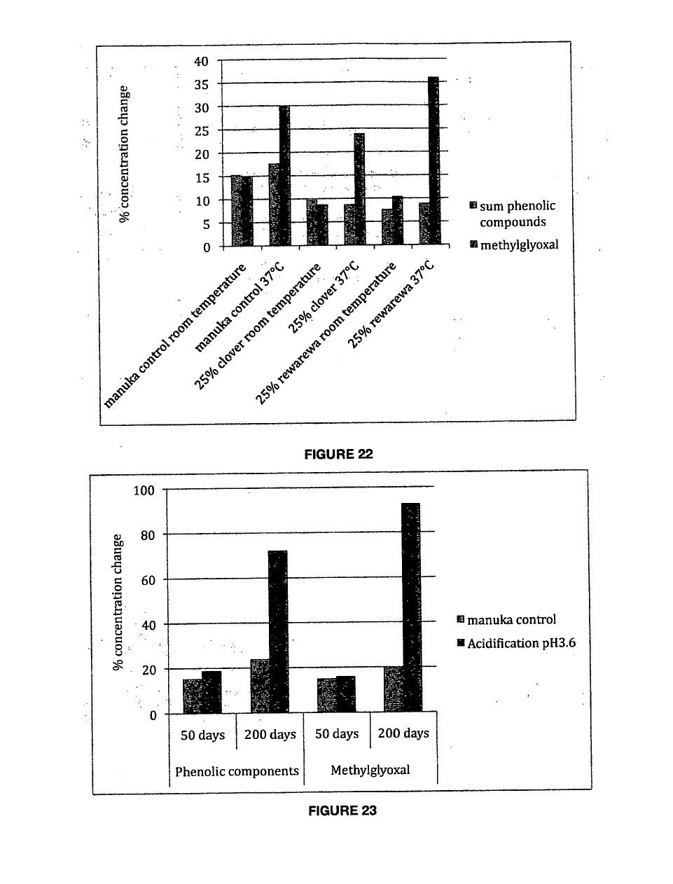

A further method found by the inventors to artificially increase the concentration of MGO in honey is to heat the honey. Unexpectedly, moderate heat had little effect on phenolic compound concentration regardless of blend. Example results are shown in FIG. 22.

Moderate heating significantly increased concentration of methylglyoxal, and this methylglyoxal concentration can be manipulated by the blend of honey utilised.

### Example 18

Acidification can be used to manipulate methylglyoxal concentration when honey is stored at room temperature.

As shown in FIG. 23, acidification dramatically increased the concentration of both phenolic compounds and MGO in honey. Acidification was demonstrated to pH 3.6.

### Example 19

In this example bacteria is added and the effect on efficacy especially with respect to the three phases of healing is observed.

Five different microbial strains were added to honeys with the aim of increasing the immune stimulatory capacity of honeys to induce an immune stimulation phase of wound healing.

The experiment was completed by producing a stock solution of 10¹⁰ cfu/ml of the following commercially available strains Lactobacillus salivarius K12, Howaru Rhamonosus, DSM Lafti B94, Staphylococcus epidermis and Micrococcus luteus. The compositions were prepared containing live culture. The bacteria were inactivated using heat (95° C.) for 15 min.

Aliquots equivalent to 109 cfu were thoroughly mixed with 10 g of honey at 30° C. The resulting mix was incubated with peripheral blood mononuclear cells taken from five different human donors and the cytokine profiles were measured.

The results observed showed that the addition of attenuated commensal or probiotic strains had a clear elevation of inflammatory cytokines in comparison to the non-treated honey control thereby increasing the immune stimulation effect.

### Example 20

A practical example is now described to produce a composition including honey with an increased level of tannin derived phenolic compounds.

In this example, a blend of manuka and kanuka honeys is produced with the aim being to elevate the total level of methoxylated phenolic compounds in the mixture. Blending alters the equilibrium as noted above developing free phenolic content.

Optionally additional flavonoid compounds may be added to the mixture for example by adding in a green tea extract, grape extract and/or a pine bark extract. These compounds may be added to accentuate the anti-inflammatory phase of healing due to their free radical scavenging activity.

### Example 21

In this example, a further practical example is provided for manufacture of a composition containing an elevated amount of tannin derived phenolic compounds.

In this example the honey may be one that already contains various phenolic compounds such as a manuka or kanuka honey and the honey is aged for a time period of up to 5 years to increase the phenolic content.

Optionally, additional anti-microbial agents may be added to the aged (or non-aged honey) including antibiotics, antiseptics and other anti-microbial agents to enhance the first anti-microbial phase of activity.

### Example 22

A further practical example is provided whereby a natural honey may be selected e.g. a manuka and kanuka honey blend. The mixture may then be heated gently to 35° C. and incubated for a time period of up to 48 hours. Tannase enzyme may then be added and optionally further steps of diluting and/or acidifying the mixture may be undertaken. The end composition has an elevated concentration of phenolic compounds then that of the native state without treatment.

### Example 23

In this practical example, a wound dressing is described tailored to be used as an initial application to an exudating wound. In order to achieve this desired activity it is preferable to accentuate the anti-microbial and immune stimulatory phases of activity. Since the dressing is likely to be reapplied within a relatively short duration, the third anti-inflammatory phase is of less importance.

The desired first and second phase activity is therefore tailored in the honey or honey analogue by increasing the peroxide activity and the MGO content in the dressing and less emphasis is placed on increasing the level of methoxylated phenolic compounds. By way of example the dressing may produced using a mixture of kanuka and clover honeys along with addition of synthetically produced MGO. An alternative approach may be to use manuka honey that has been heated or acidified on order to elevate the concentration of MGO in the manuka honey.

### Example 24

In this example, a wound dressing is developed in order to accentuate the third anti-inflammatory phase and minimise the anti-microbial phase. An example of when this dressing might be used is when a dressing is re-applied to an already healing wound.

To tailor a dressing to achieve this effect, the dressing is produced by taking a honey or honey analogue, avoiding the presence of MGO and increasing the predominance of phenolic compounds, in particular methoxylated phenolic compounds or more specifically, phenolic compounds including phenyllactic acid, methoxylated phenyllactic acid, methoxylated benzoic acids, syringic acid, methyl syringate, and/or isomeric forms of methyl syringate.

This may be achieved by selection of a low UMF manuka honey or use of honeys with no UMF activity but high phenolic content e.g. kanuka honey. The chosen honey may also be processed by dilution, tannase addition and/or dilution to further accentuate the phenolic compound effects.

### Example 25

In this example, a nutritional supplement is described to assist with gut health an in particular to control H. pylori growth and damage in the gut. The supplement is a tablet or capsule of functional food containing a honey or honey analogue tailored to elicit a three phase effect of healing including an anti-microbial phase, an immune stimulation phase and an anti-inflammatory phase. The honey or honey analogue is tailored to include a variety of phenolic compounds including methoxylated phenolic compounds.

### Example 26

A further maintenance example is described. In honey processing a very good potency honey may be produced and through undesirable handling techniques, the honey may lose potency.

To address this loss, a honey may be sourced initially from plants that produce high levels of phenolics and methoxylated phenolics in the plant nectar. Subsequent processing may then be controlled to avoid blending, heating, dilution and/or acidity. The honey may also be aged to fully develop the free phenolic content.

### Example 27

As noted above, adding fungal material may influence the degree of immune stimulation in the second phase of healing.

In this example, a composition is produced that accentuates this immune stimulation phase. The composition is produced in a similar manner to other compositions described above however, in order to manipulate and enhance the second immune stimulation effect, fungal material in the form of cell wall complex carbohydrates is blended (fortified) into the honey or honey analogues.

Besides blending in cell wall complex carbohydrates, it is also possible to generate fungal cell wall material by partially fermenting the honey. Honey is typically collected and stored in a metal drum. The drum may be inoculated with yeast and left to ferment in a similar manner to wine making for a period of time. By this process, the yeast multiplies (as does fungal cell wall materials). The fermentation process may then be stopped at any stage by heat treatment and, for medical uses the honey then irradiated to ensure all active yeast is killed. The resulting honey is therefore manipulated via a fermentation process to have fungal material therein.

Aspects of the methods described herein have been described by way of example only and it should be appreciated that modifications and additions may be made thereto without departing from the scope of the claims herein.

What is claimed is:

1. A composition including honey or a honey analogue wherein the honey or honey analogue is artificially manipulated and/or fortified to include at least 5 mg/kg of tannin derived phenolic compounds.

2. The composition as claimed in claim 1 wherein the composition includes at least 5-10,000 mg/kg tannin derived phenolic compounds.

3. The composition as claimed in claim 1 or 2 wherein the phenolic compounds are methoxylated.

4. The composition as claimed in claim 3 wherein the composition includes at least 150 mg/kg methoxylated phenolic compounds.

5. The composition as claimed in any one of the above claims wherein the phenolic compounds are selected from the group consisting of: phenyllactic acid, methoxylated phenyllactic acid, methoxylated benzoic acids, syringic acid, methyl syringate, isomeric forms of methyl syringate, and combinations thereof.

6. The composition as claimed in claim 5 wherein the methoxylated derivatives of benzoic acid are selected from the group consisting of: 2-methoxybenzoic acid, 4-methoxybenzoic acid, trimethoxybenzoic acid and combinations thereof.

7. The composition as claimed in any one of the above claims wherein the composition includes a blend of honey or honey analogues.

8. The composition as claimed in any one of the above claims wherein the honey or honey analogue is manipulated by aging the honey or honey analogue for a time period of at least 1 year.

9. The composition as claimed in any one of the above claims wherein the composition is manipulated by heating.

10. The composition as claimed in any one of the above claims wherein the composition is manipulated and/or fortified by addition of further compounds selected from: tannase enzymes; aqueous diluting agent, commensal bacteria, commensal fungi, flavonoid sources, anti-microbial agents, synthetic anti-inflammatory agents, MGO, acidifying agent, and combinations thereof.

11. The composition as claimed in any one of the above claims wherein the composition is fortified by the inclusion of fungal material.

12. The composition as claimed in any one of the above claims wherein the composition is manipulated to include an artificially elevated level of fungal material by the honey or honey analogue being at least partially fermented with a yeast inoculum.

13. The composition as claimed in claim 11 or claim 12 wherein the fungal material includes complex carbohydrate compounds associated with the cell wall of fungal cells.

14. The composition as claimed in any one of the above claims wherein the composition is formed into a wound dressing by further manufacture into formulations selected from: a cream, an ointment, a gel, a putty, a fibre dressing with the honey impregnated into or around the fibre, a fibre dressing with the honey enclosed within one or more fibre layers, and combinations thereof.

15. Use of the composition as claimed in any one of claims 1 to 14 in a wound dressing.

16. Use of the composition as claimed in any one of claims 1 to 14 in a nutritional supplement.

17. A method of maintaining or increasing the medical and/or nutritional potency of a honey or honey analogue composition by the steps of:
(a) selecting one or more honeys or honey analogues;
(b) artificially manipulating and/or fortifying the honey or honey analogue to increase the concentration of at least one tannin derived phenolic compound in the honey(s) or honey analogue(s) to a level of 5 mg/kg or higher.

18. The method as claimed in claim 17 wherein step (b) increases the concentration of phenolic compounds to a level of 5 to 10,000 mg/kg or higher.

19. The method as claimed in claim 17 or claim 18 wherein the phenolic compounds include at least 150 mg/kg methoxylated phenolic compounds.

20. The method as claimed in any one of claims 17 to 19 wherein the phenolic compounds are selected from the group consisting of: phenyllactic acid, methoxylated phenyllactic acid, methoxylated benzoic acids, syringic acid, methyl syringate, isomeric forms of methyl syringate, and combinations thereof.

21. The method as claimed in claim 19 wherein the methoxylated derivatives of benzoic acid are selected from the group consisting of: 2-methoxybenzoic acid, 4-methoxybenzoic acid, trimethoxybenzoic acid, and combinations thereof.

22. The method as claimed in any one of claims 17 to 21 wherein manipulation includes blending of different honey types and/or honey analogues.

23. The method as claimed in any one of claims 17 to 22 wherein manipulation includes aging the honey or honey analogue for a time period of at least 1 year.

24. The method as claimed in any one of claims 17 to 23 wherein manipulation includes heating the honey or honey analogue.

25. The composition as claimed in claim 24 wherein the heating temperature is less than 40° C.

26. The composition as claimed in any one of claims 17 to 25 wherein manipulation includes adding tannase enzymes to the honey or honey analogue.

27. The composition as claimed in any one of claims 17 to 25 wherein manipulation includes adding an aqueous diluent to the honey or honey analogue.

28. The composition as claimed in any one of claims 17 to 25 wherein fortification includes adding MGO.

29. The composition as claimed in any one of claims 17 to 25 wherein manipulation includes acidifying the honey or honey analogue.

30. The composition as claimed in any one of claims 17 to 25 wherein the composition is fortified by the inclusion of fungal material.

31. The composition as claimed in any one of claims 17 to 25 wherein the composition is manipulated to include an artificially elevated level of fungal material by the honey or honey analogue being at least partially fermented with a yeast inoculum.

32. The composition as claimed in claim 30 or claim 31 wherein the fungal material includes complex carbohydrate compounds associated with the cell wall of fungal cells.

33. The composition as claimed in any one of claims 17 to 32 wherein the composition is formed into a wound dressing by further manufacture into formulations selected from: a cream, an ointment, a gel, a putty, a fibre dressing with the honey impregnated into or around the fibre, a fibre dressing with the honey enclosed within one or more fibre layers, and combinations thereof.

34. A method of treatment of a wound on a non-human animal by application of a wound dressing containing a honey or a honey analogues wherein the honey or honey analogue has been artificially manipulated and/or fortified to include at least 5 mg/kg of tannin derived phenolic compounds and wherein, on application to a wound, the composition induces three phases of healing including:
(a) an anti-microbial phase;
(b) an immune stimulation phase; and,
(c) an anti-inflammatory phase.

35. The method as claimed in claim 34 wherein the anti-microbial phase includes actions selected from the group consisting of: lowering of the pH, elevation of the osmolarity in the wound area, release of hydrogen peroxide, slowing microbial growth, delaying the onset of microbial growth, stopping microbial growth, killing existing microbes, and combinations thereof.

34. The method as claimed in claim 34 or claim 35 wherein the immune stimulation phase includes production of pro-inflammatory cytokines selected from the group consisting of: TNFα, IL-1. IL1β, IL-6, IL-10, 10F-α, and combinations thereof.

37. The method as claimed in any one of claims 34 to 36 wherein the immune stimulation phase includes debriding action associated by an elevation of MMP protease enzyme activity.

38. The method as claimed in any one of claims 34 to 36 wherein the immune stimulation phase occurs after the first phase is complete or happens concurrently with the first phase.

39. The method as claimed in any one of claims 34 to 38 wherein the anti-inflammatory phase includes one or more actions selected from the group consisting of: reduction in inflammation, an inhibition of proteolytic tissue degrading enzymes (MMP-proteases), reduction in the levels of free radicals (quenching of peroxide levels), an increase of glutathione levels, induction of phase II enzyme inducer activity, and combinations thereof.

40. The method as claimed in any one of claims 34 to 39 wherein the dressing is artificially manipulated and/or fortified to accentuate step (a), an anti-microbial phase.

41. The method as claimed in any one of claims 34 to 39 wherein the dressing is artificially manipulated and/or fortified to accentuate step (b), an immune stimulation phase.

42. The method as claimed in any one of claims 34 to 39 wherein the dressing is artificially manipulated and/or fortified to accentuate step (c), an anti-inflammatory phase.

43. Use of honey or a honey analogue based composition that has been artificially manipulated and/or fortified to include at least 5 mg/kg of tannin derived phenolic compounds in the manufacture of a wound dressing for the treatment of a topical wound on an animal in need thereof and wherein, on application to a wound, the composition induces three phases of healing including:
(a) an anti-microbial phase;
(b) an immune stimulation phase; and,
(c) an anti-inflammatory phase.

44. The use as claimed in claim 43 wherein the anti-microbial phase includes actions selected from the group consisting of: lowering of the pH, elevation of the osmolarity in the wound area, release of hydrogen peroxide, slowing microbial growth, delaying the onset of microbial growth, stopping microbial growth, killing existing microbes, and combinations thereof.

45. The use as claimed in claim 43 or claim 44 wherein the immune stimulation phase includes production of pro-inflammatory cytokines selected from the group consisting of: TNFα, IL-1. IL-1β, IL-6, IL-10, 10F-α, and combinations thereof.

46. The use as claimed in any one of claims 43 to 45 wherein the immune stimulation phase includes debriding action associated by an elevation of MMP protease enzyme activity.

47. The use as claimed in any one of claims 43 to 46 wherein the immune stimulation phase occurs after the first phase is complete or happens concurrently with the first phase.

48. The use as claimed in any one of claims 43 to 47 wherein the anti-inflammatory phase includes one or more actions selected from the group consisting of reduction in inflammation, an inhibition of proteolytic tissue degrading enzymes (MMP-proteases), reduction in the levels of free radicals (quenching of peroxide levels), an increase of glutathione levels, induction of phase II enzyme inducer activity, and combinations thereof.

49. The use as claimed in any one of claims 43 to 48 wherein the dressing is artificially manipulated and/or fortified to accentuate step (a), an anti-microbial phase.

50. The use as claimed in any one of claims 43 to 48 wherein the dressing is artificially manipulated and/or fortified to accentuate step (b), an immune stimulation phase.

51. The use as claimed in any one of claims 43 to 48 wherein the dressing is artificially manipulated and/or fortified to accentuate step (c), an anti-microbial phase.
